(19) United States
(12) Patent Application Publication
(10) Pub. No.: US 2004/0103448 A1
(43) Pub. Date: May 27, 2004

(54) Methods for inducing in vivo proliferation and migration of transplanted progenitor cells in the brain

(76) Inventor: Anders Bjorklund, Lund (SE)

Correspondence Address: MINTZ, LEVIN, COHN, FERRIS, GLOVSKY, AND POPEO, P.C., ONE FINANCIAL CENTER, BOSTON, MA, 02111, US

(21) Appl. No.: 10/713,373

(22) Filed: Nov. 13, 2003

Related U.S. Application Data
(63) Continuation of application No. 09/693,043, filed on Oct. 20, 2000.
(63) Continuation-in-part of application No. 09/339,093, filed on Jun. 23, 1999, now abandoned.
Application No. 09/339,093 is a division of application No. 08/926,313, filed on Sep. 5, 1997, now Pat. No. 5,968,829.
(60) Provisional application No. 60/160,553, filed on Oct. 20, 1999.

(30) Foreign Application Priority Data
Sep. 4, 1998 (WO) PCT/US98/18597

(51) Int. Cl. A01K 67/00; C12N 5/08

(52) U.S. Cl. 800/9; 435/368

(57) ABSTRACT

The present invention provides methods of inducing in vivo migration and proliferation of progenitor cells transplanted to the brain. Isolation, characterization, proliferation, differentiation and transplantation of mammalian neural stem cells are also disclosed.

### RELATED APPLICATIONS

This application is a continuation of U.S. Ser. No. 09/693,043, filed Oct. 20, 2003, which claims priority to U.S. Ser. No. 60/160,553, filed Oct. 20, 1999; and which is a continuation-in-part of U.S. Ser. No. 09/339,093, filed Jun. 23, 1999, which is a divisional of U.S. Ser. No. 08/926,313, filed Sep. 5, 1997, now issued as U.S. Pat. No. 5,968,829; and which is a continuation-in-part of U.S. Ser. No. 09/486,302, filed Feb. 24, 2000 and PCT/US98/18597, filed Sep. 4, 1998; the teachings of each of which are incorporated herein by reference in their entirety.

### FIELD OF THE INVENTION

This invention relates to isolation of human central nervous system stem cells, and methods and media for proliferating, differentiating and transplanting them.

### BACKGROUND OF THE INVENTION

During development of the central nervous system (“CNS”), multipotent precursor cells, also known as neural stem cells, proliferate, giving rise to transiently dividing progenitor cells that eventually differentiate into the cell types that compose the adult brain. Stem cells (from other tissues) have classically been defined as having the ability to self-renew (i.e., form more stem cells), to proliferate, and to differentiate into multiple different phenotypic lineages. In the case of neural stem cells this includes neurons, astrocytes and oligodendrocytes. For example, Potten and Loeffler (Development, 110:1001, 1990) define stem cells as “undifferentiated cells capable of: (a) proliferation, (b) self-maintenance, (c) the production of a large number of differentiated functional progeny, (d) regenerating the tissue after injury, and (e) a flexibility in the use of these options.”

These neural stem cells have been isolated from several mammalian species, including mice, rats, pigs and humans. See, e.g., WO 93/01275, WO 94/09119, WO 94/10292, WO 94/16718 and Cattaneo et al., Mol. Brain Res., 42, pp. 161-66 (1996), all herein incorporated by reference.

Human CNS neural stem cells, like their rodent homologues, when maintained in a mitogen-containing (typically epidermal growth factor or epidermal growth factor plus basic fibroblast growth factor), serum-free culture medium, grow in suspension culture to form aggregates of cells known as “neurospheres.” Human neural stem cells have been shown to have doubling rates of about 30 days. See, e.g., Cattaneo et al., Mol. Brain Res., 42, pp. 161-66 (1996). Upon removal of the mitogen(s) and provision of a substrate, the stem cells differentiate into neurons, astrocytes and oligodendrocytes. In the prior art, the majority of cells in the differentiated cell population have been identified as astrocytes, with very few neurons (<10%) being observed.

There has been recent interest in a population of cells within the adult central nervous system (CNS) which exhibit stem cell properties, in their ability to self-renew and to produce the differentiated mature cell phenotypes of the adult CNS. In vivo intraventricular infusion of epidermal growth factor (EGF) results in proliferation of at least two different populations of cells found within the periventricular region, both a constitutively dividing population of neural progenitor cells and a relatively quiescent population of cells with stem cell-like properties. See, e.g., Craig et al., Journal ofNeuroscience 16, pp. 2649-2658 (1996). When stimulated to divide by the presence of EGF, these endogenous stem/progenitor cells do not follow the normal migration pattern along the rostral migratory pathway towards the olfactory bulb to regenerate neurons (See, e.g., Lois et al., Science 264, pp. 1145-1148 (1994); Luskin, Neuron 11, pp. 173-189 (1993)), but rather migrate laterally into the surrounding parenchyma of the striatum, cortex and septum where they differentiate into glia and reside in a satellite position to the intrinsic neurons of the adult CNS. See, e.g., Kuhn et al., The Journal ofNeuroscience 17, pp. 5820-5829 (1997).

Cell transplantation offers a possibility to provide new cellular elements in response to damage of the adult mammalian brain, as a means to modify the brain's response to injury or degeneration by implantation of new neurons or glia. Although many neurotrophic factors have been shown to affect the growth, differentiation potential, and survival of progenitor cells in vitro (see, e.g., Ahmed et al., Journal ofNeuroscience 15, pp. 5765-5778 (1995)), problems associated with the limited migration, proliferation, and differentiation of transplanted cells in vivo remain.

There remains a need to increase the rate of proliferation of neural stem cell cultures. There also remains a need to increase the number of neurons in the differentiated cell population. There further remains a need to improve the viability of neural stem cell grafts upon implantation into a host, including a need to improve the in vivo proliferation and directed migration of undifferentiated progenitor cells after transplantation to the brain.

### SUMMARY OF THE INVENTION

The invention provides methods for inducing the in vivo migration and proliferation of progenitor cells transplanted to the brain. In one embodiment, there is provided a method for inducing in vivo migration of progenitor cells transplanted to the brain by transplanting progenitor cells to a first locus of the brain of a subject, and inducing in vivo migration of the transplanted cells by infusing a mitogenic growth factor at a second locus of the brain. In some preferred embodiments, the first locus is in the striatum of the brain, and the second locus at which a mitogenic growth factor is infused is the lateral ventricle of the brain. In other preferred embodiments, a mitogenic growth factor infusion induces migration towards the second locus (e.g., locus of infusion) but does not induce differentiation of the progenitor cells.

In another embodiment, there is provided a method for inducing in vivo proliferation of progenitor cells transplanted to the brain by transplanting progenitor cells to a locus of the brain of a subject, and inducing in vivo proliferation of the transplanted cells by infusing a mitogenic growth factor at or near the locus of transplantation. In some preferred embodiments, the locus of transplantation is in the striatum of the brain, and a mitogenic growth factor is infused in the lateral ventricle of the brain. In other embodiments of the methods of the invention, the progenitor cells are mammalian embryonic progenitor cells, and the progenitor cells are cultured in media containing a mitogenic growth factor prior to transplantation.

The invention further provides novel human central nervous system stem cells, and methods and media for proliferating, differentiating and transplanting them. In one embodiment, this invention provides novel human stem cells with a doubling rate of between 5-10 days, as well as defined growth media for prolonged proliferation of human neural stem cells. In another embodiment, this invention provides a defined media for differentiation of human neural stem cells so as to enrich for neurons, oligodendrocytes, astrocytes, or a combination thereof. The invention also provides differentiated cell populations of human neural stem cells that provide previously unobtainable large numbers of neurons, as well as astrocytes and oligodendrocytes. This invention also provides novel methods for transplanting neural stem cells that improve the viability of the graft upon implantation in a host.

Methods of the present invention can be used in preparation of a medicament for inducing in vivo proliferation and migration of transplanted progenitor cells in the brain.

### BRIEF DESCRIPTION OF THE DRAWINGS

FIG. 1 shows a representation of spheres of proliferating 9FBr human neural stem cells (passage 6) derived from human forebrain tissue.

FIG. 2, Panel A, shows a growth curve for a human neural stem cell line designated 6.5Fbr cultured in (a) defined media containing EGF, FGF and leukemia inhibitory factor (“LIF”) (shown as closed diamonds), and (b) the same media but without LIF (shown as open diamonds); Panel B shows a growth curve for a human neural stem cell line designated 9Fbr cultured in (a) defined media containing EGF, FGF and LIF (shown as closed diamonds), and (b) the same media but without LIF (shown as open diamonds); Panel C shows a growth curve for a human neural stem cell line designated 9.5Fbr cultured in (a) defined media containing EGF, FGF and LIF (shown as closed diamonds), and (b) the same media but without LIF (shown as open diamonds); Panel D shows a growth curve for a human neural stem cell line designated 10.5Fbr cultured in (a) defined media containing EGF, FGF and leukemia inhibitory factor (“LIF”) (shown as closed diamonds), and (b) the same media but without LIF (shown as open diamonds).

FIG. 3 shows a growth curve for a human neural stem cell line designated 9Fbr cultured in (a) defined media containing EGF and basic fibroblast growth factor (“bFGF”) (shown as open diamonds), and (b) defined media with EGF but without bFGF (shown as closed diamonds).

FIG. 4 shows a graph of cell number versus days in culture for a Mx-1 conditionally immortalized human glioblast line derived from a human neural stem cell line. The open squares denote growth in the presence of interferon; the closed diamonds denote growth in the absence of interferon.

FIG. 5 shows images of rat brain after transplantation of progenitor cells. All transplanted cells are identified by the antigen M2 (red). Panels A-C show low power images the medial striatum labeled with M2 (red) and BrdU (green), from A) the contralateral side of an EGF-infused animal, B) the transplant core of a vehicle-infused animal and C) the transplant core of an EGF-infused animal. (LV=lateral ventricle). Panels D-G indicate co-labeling with M2 (red), GFAP (green) and BrdU (blue) of D) vehicle-infused, E-G) EGF-infused animal, with F) high power within the transplant core and G) high power within the region between the transplant and lateral ventricle. Arrowheads indicate double-labeled BrdU/M2 cells and arrows indicate double-labeled BrdU/GFAP cells. Panels H-K show double labeling with M2 (red) and vimentin (VIM; green) of H), vehicle-infused and I-K) EGF-infused with J) high power of the region between transplant core and lateral ventricle and K) increased expression of VIM in the SVZ. Panel L shows triple labeling with M2 (red), nestin (green), and BrdU (blue) of an EGF-infused animal with M) a high power image of the region between the transplant core and the lateral ventricle. Arrowheads indicate BrdUIM2 double-labeled cells and arrows indicate BrdU and nestin colocalization. Scale bar in M: A-C=300 μm; D,E,H,I=400 μm; F,G,J,M=15 μm; K,L=200 μm.

FIG. 6 is a camera lucida drawing of a series of 1:8 coronal sections in an A) vehicle-infused and B) EGF-infused animal showing the distribution of M2-positive profiles throughout the transplant and adjacent parenchyma. CC: corpus callosum; Str: striatum; LV: lateral ventricle; SM: stria medullaris. Asterisk indicates the level of cannulae placement and associated damage to the cortex.

FIG. 7 shows images of the distribution of ³H-thymidine labeled cells (silver grains) and BrdU-labeled cells within the region between the transplant core and the lateral ventricle, in a A) EGF-infused and B) vehicle-infused animal. Scattered ³H-thymidine positive cells are indicated with arrows, and the occasional BrdU/³H-thymidine double-labeled cell is marked with an arrowhead (insert in A). Note the lack of ³H-thymidine labeled cells in B. Scale bar in B=80 μm

FIG. 8 shows images of β-galactosidase (βgal) labeling of a typical transplant. A) Within the transplant core, only immature βgal-positive cells were observed. B and C) Occasional cells were found scattered within the striatum (B) or corpus callosum (C) and had the identity of immature oligodendrocytes. T: transplant; Ctx: cortex; CC: corpus callosum; Str: striatum. Scale bar in A=50 cm; and C (for B and C)=20 μm.

### DETAILED DESCRIPTION OF THE INVENTION

This invention relates to isolation, characterization, proliferation, differentiation and transplantation of CNS neural stem cells. The invention further relates to inducing the in vivo migration or proliferation of progenitor cells transplanted to the brain.

The neural stem cells described and claimed in the applications may be proliferated in suspension culture or in adherent culture. When the neural stem cells of this invention are proliferating as neurospheres, human nestin antibody may be used as a marker to identify undifferentiated cells. The proliferating cells show little GFAP staining and little β-tubulin staining (although some staining might be present due to diversity of cells within the spheres).

When differentiated, most of the cells lose their nestin positive immunoreactivity. In particular, antibodies specific for various neuronal or glial proteins may be employed to identify the phenotypic properties of the differentiated cells. Neurons may be identified using antibodies to neuron specific enolase (“NSE”), neurofilament, tau, beta-tubulin, or other known neuronal markers. Astrocytes may be identified using antibodies to glial fibrillary acidic protein (“GFAP”), or other known astrocytic markers. Oligodendrocytes may be identified using antibodies to galactocerebroside, O4, myelin basic protein (“MBP”) or other known oligodendrocytic markers. Glial cells in general may be identified by staining with antibodies, such as the M2 antibody, or other known glial markers.

In one embodiment the invention provides novel human CNS stem cells isolated from the forebrain. Four neural stem cell lines have been isolated from human forebrain, all of which exhibit neural stem cell properties; namely, the cells are self renewing, the cells proliferate for long periods in mitogen containing serum free medium, and the cells, when differentiated, comprise a cell population of neurons, astrocytes and oligodendrocytes. These cells are capable of doubling every 5-10 days, in contrast with the prior art diencephalon-derived human neural stem cells. Reported proliferation rates of diencephalon-derived human neural stem cells approximate one doubling every 30 days. See Cattaneo et al., Mol. Brain Res., 42, pp. 161-66 (1996).

Any suitable tissue source may be used to derive the neural stem cells of this invention. Neural stem cells can be induced to proliferate and differentiate either by culturing the cells in suspension or on an adherent substrate. See, e.g., U.S. Pat. Nos. 5,750,376 and 5,753,506 (both incorporated herein by reference in their entirety), and prior art medium described therein. Both allografts and autografts are contemplated for transplantation purposes.

This invention also provides a novel growth media for proliferation of neural stem cells. Provided herein is a serum-free or serum-depleted culture medium for the short term and long term proliferation of neural stem cells.

A number of serum-free or serum-depleted culture media have been developed due to the undesirable effects of serum which can lead to inconsistent culturing results. See, e.g., WO 95/00632 (incorporated herein by reference), and prior art medium described therein.

Prior to development of the novel media described herein, neural stem cells have been cultured in serum-free media containing epidermal growth factor (“EGF”) or an analog of EGF, such as amphiregulin or transforming growth factor alpha (“TGF-α”), as the mitogen for proliferation. See, e.g., WO 93/01275, WO 94/16718, both incorporated herein by reference. Further, basic fibroblast growth factor (“bFGF”) has been used, either alone, or in combination with EGF, to enhance long term neural stem cell survival.

The improved medium according to this invention, which contains leukemia inhibitory factor (“LIF”), markedly and unexpectedly increases the rate of proliferation of neural stem cells, particularly human neural stem cells.

The growth rates of the forebrain-derived stem cells described herein were compared in the presence and absence of LIF. Unexpectedly, LIF was found to dramatically increase the rate of cellular proliferation in almost all cases.

The medium according to this invention comprises cell viability and cell proliferation effective amounts of the following components:

(a) a standard culture medium being serum-free (containing 0-0.49% serum) or serum-depleted (containing 0.5-5.0% serum), known as a “defined” culture medium, such as Iscove's modified Dulbecco's medium (“IMDM”), RPMI, DMEM, Fischer's, alpha medium, Leibovitz's, L-15, NCTC, F-10, F-12, MEM and McCoy's;

(b) a suitable carbohydrate source, such as glucose;

(c) a buffer such as MOPS, HEPES or Tris, preferably HEPES;

(d) a source of hormones including insulin, transferrin, progesterone, selenium, and putrescine;

(e) one or more growth factors that stimulate proliferation of neural stem cells, such as EGF, bFGF, PDGF, NGF, and analogs, derivatives and/or combinations thereof, preferably EGF and bFGF in combination; and

(f) LIF.

Standard culture media typically contains a variety of essential components required for cell viability, including inorganic salts, carbohydrates, hormones, essential amino acids, vitamins, and the like. Preferably, DMEM or F-12 is used as the standard culture medium, most preferably a 50/50 mixture of DMEM and F-12. Both media are commercially available (DMEM-Gibco 12100-046; F-12- Gibco 21700-075). A premixed formulation is also commercially available (N-2-Gibco 17502-030). It is advantageous to provide additional glutamine, preferably at about 2 mM. It is also advantageous to provide heparin in the culture medium. Preferably, the conditions for culturing should be as close to physiological as possible. The pH of the culture medium is typically between 6-8, preferably about 7, most preferably about 7.4. Cells are typically cultured between 30-40° C., preferably between 32-38° C., most preferably between 35-37° C. Cells are preferably grown in 5% CO₂. Cells are preferably grown in suspension culture.

In one exemplary embodiment, the neural stem cell culture comprises the following components in the indicated concentrations:

| COMPONENT | FINAL CONCENTRATION |
|---|---|
| 50/50 mix of DMEM/F-12 | 0.5-2.0 X, preferably 1X |
| glucose | 0.2-1.0%, preferably 0.6% w/v |
| glutamine | 0.1-10 mM, preferably 2 mM |
| NaHCO₃ | 0.1-10 mM, preferably 3 mM |
| HEPES | 0.1-10 mM, preferably 5 mM |
| apo-human transferrin (Sigma T-2252) | 1-1000 μg/ml, |
| | preferably 100 μg/ml |
| human insulin (Sigma I-2767) | 1-100, preferably 25 μg/ml |
| putrescine (Sigma P-7505) | 1-500, preferably 60 μM |
| selenium (Sigma S-9133) | 1-100, preferably 30 nM |
| progesterone (Sigma P-6149) | 1-100, preferably 20 nM |
| human EGF (Gibco 13247-010) | 0.2-200, preferably 20 ng/ml |
| human bFGF (Gibco 13256-029) | 0.2-200, preferably 20 ng/ml |
| human LIF (R&D Systems 250-L) | 0.1-500, preferably 10 ng/ml |
| heparin (Sigma H-3149) | 0.1-50, preferably 2 μg/ml |
| CO₂ | preferably 5% |

Serum albumin may also be present in the instant culture medium—although the present medium is generally serum-depleted or serum-free (preferably serum-free), certain serum components which are chemically well defined and highly purified (>95%), such as serum albumin, may be included.

The human neural stem cells described herein may be cryopreserved according to routine procedures. Preferably, about one to ten million cells are cryopreserved in “freeze” medium that consists of proliferation medium (absent the growth factor mitogens), 10% BSA (Sigma A3059) and 7.5% DMSO. Cells are centrifuged. Growth medium is aspirated and replaced with freeze medium. Cells are resuspended gently as spheres, not as dissociated cells. Cells are slowly frozen, by, e.g., placing in a container at −80° C. Cells are thawed by swirling in a 37° C. bath, resuspended in fresh proliferation medium, and grown as usual.

In another embodiment, this invention provides a differentiated cell culture containing previously unobtainable large numbers of neurons, as well as astrocytes and oligodendrocytes. In the prior art, typically the differentiated human diencephalon-derived neural stem cell cultures formed very few neurons (i.e., less than 5-10%). According to this methodology, neuron concentrations of between 20% and 35% (and much higher in other cases) are routinely achieved in differentiated human forebrain-derived neural stem cell cultures. This is highly advantageous, as it permits enrichment of the neuronal population prior to implantation in the host in disease indications where neuronal function has been impaired or lost.

Further, according to the methods of this invention, differentiated neural stem cell cultures have been achieved that are highly enriched in GABAergic neurons. Such GABAergic neuron enriched cell cultures are particularly advantageous in the potential therapy of excitotoxic neurodegenerative disorders, such as Huntington's disease or epilepsy.

In order to identify the cellular phenotype either during proliferation or differentiation of the neural stem cells, various cell surface or intracellular markers may be used.

When the neural stem cells of this invention are proliferating as neurospheres, human nestin antibody can be used as a marker to identify undifferentiated cells. The proliferating cells should show little GFAP staining and little β-tubulin staining (although some staining might be present due to diversity of cells within the spheres).

When differentiated, most of the cells lose their nestin positive immunoreactivity. In particular, antibodies specific for various neuronal or glial proteins may be employed to identify the phenotypic properties of the differentiated cells. Neurons may be identified using antibodies to neuron specific enolase (“NSE”), neurofilament, tau, β-tubulin, or other known neuronal markers. Astrocytes may be identified using antibodies to glial fibrillary acidic protein (“GFAP”), or other known astrocytic markers. Oligodendrocytes may be identified using antibodies to galactocerebroside, O4, myelin basic protein (“MBP”) or other known oligodendrocytic markers.

It is also possible to identify cell phenotypes by identifying compounds characteristically produced by those phenotypes. For example, it is possible to identify neurons by the production of neurotransmitters such as acetylcholine, dopamine, epinephrine, norepinephrine, and the like.

Specific neuronal phenotypes can be identified according to the specific products produced by those neurons. For example, GABAergic neurons may be identified by their production of glutamic acid decarboxylase (“GAD”) or GABA. Dopaminergic neurons may be identified by their production of dopa decarboxylase (“DDC”), dopamine or tyrosine hydroxylase (“TH”). Cholinergic neurons may be identified by their production of choline acetyltransferase (“ChAT”). Hippocampal neurons may be identified by staining with NeuN. It will be appreciated that any suitable known marker for identifying specific neuronal phenotypes may be used.

The human neural stem cells described herein can be genetically engineered or modified according to known methodology. The term “genetic modification” refers to the stable or transient alteration of the genotype of a cell by intentional introduction of exogenous DNA. DNA may be synthetic, or naturally derived, and may contain genes, portions of genes, or other useful DNA sequences. The term “genetic modification” is not meant to include naturally occurring alterations such as that which occurs through natural viral activity, natural genetic recombination, or the like.

A gene of interest (i.e., a gene that encodes a biologically active molecule) can be inserted into a cloning site of a suitable expression vector by using standard techniques. These techniques are well known to those skilled in the art. See, e.g., WO 94/16718, incorporated herein by reference.

The expression vector containing the gene of interest may then be used to transfect the desired cell line. Standard transfection techniques such as calcium phosphate co-precipitation, DEAE-dextran transfection, electroporation, biolistics, or viral transfection may be utilized. Commercially available mammalian transfection kits may be purchased from e.g., Stratagene. Human adenoviral transfection may be accomplished as described in Berg et al. Exp. Cell Res., 192, pp. (1991). Similarly, lipofectamine-based transfection may be accomplished as described in Cattaneo, Mol. Brain Res., 42, pp. 161-66 (1996).

A wide variety of host/expression vector combinations may be used to express a gene encoding a biologically active molecule of interest. See, e.g., U.S. Pat. No. 5,545,723, herein incorporated by reference, for suitable cell-based production expression vectors.

Increased expression of the biologically active molecule can be achieved by increasing or amplifying the transgene copy number using amplification methods well known in the art. Such amplification methods include, e.g., DHFR amplification (see, e.g., Kaufman et al., U.S. Pat. No. 4,470,461) or glutamine synthetase (“GS”) amplification (see, e.g., U.S. Pat. No. 5,122,464, and European published application EP 338,841), all herein incorporated by reference.

In another embodiment, the genetically modified neural stem cells are derived from transgenic animals.

When the neural stem cells are genetic modified for the production of a biologically active substance, the substance will preferably be useful for the treatment of a CNS disorder. To this end, genetically modified neural stem cells can be produced that are capable of secreting a therapeutically effective biologically active molecule in patients. Further contemplated is the production of a biologically active molecule with growth or trophic effect on the transplanted neural stem cells. Further contemplated is inducing differentiation of the cells towards neural cell lineages. The genetically modified neural stem cells thus provide cell-based delivery of biological agents of therapeutic value.

The neural stem cells described herein, and their differentiated progeny may be immortalized or conditionally immortalized using known techniques. Conditional immortalization of stem cells is preferred, and most preferably conditional immortalization of their differentiated progeny. Among the conditional immortalization techniques contemplated are Tet-conditional immortalization (see WO 96/31242, incorporated herein by reference), and Mx-1 conditional immortalization (see WO 96/02646, incorporated herein by reference).

This invention also provides methods for differentiating neural stem cells to yield cell cultures enriched with neurons to a degree previously unobtainable. According to one protocol, the proliferating neurospheres are induced to differentiate by removal of the growth factor mitogens and LIF, and provision of 1% serum, a substrate and a source of ionic charges (e.g., glass cover slip covered with poly-omithine or extracellular matrix components). The preferred base medium for this differentiation protocol, excepting the growth factor mitogens and LIF, is otherwise the same as the proliferation medium. This differentiation protocol produces a cell culture enriched in neurons. According to this protocol, neuron concentrations of between 20% and 35% have been routinely achieved in differentiated human forebrain-derived neural stem cell cultures.

According to a second protocol, the proliferating neurospheres are induced to differentiate by removal of the growth factor mitogens, and provision of 1% serum, a substrate and a source of ionic charges (e.g., glass cover slip covered with poly-omithine or extracellular matrix components), as well as a mixture of growth factors including PDGF, CNTF, IGF-1, LIF, forskolin, T-3 and NT-3. The cocktail of growth factors may be added at the same time as the neurospheres are removed from the proliferation medium, or may be added to the proliferation medium and the cells pre-incubated with the mixture prior to removal from the mitogens. This protocol produces a cell culture highly enriched in neurons and enriched in oligodendrocytes. According to this protocol, neuron concentrations of higher than 35% have been routinely achieved in differentiated human forebrain-derived neural stem cell cultures.

The presence of bFGF in the proliferation media unexpectedly inhibits oligodendrocyte differentiation capability. bFGF is trophic for the oligodendrocyte precursor cell line. Oligodendrocytes are induced under differentiation conditions when passaged with EGF and LIF in proliferating media, without bFGF.

The human stem cells of this invention have numerous uses, including for drug screening, diagnostics, genomics and transplantation. Stem cells can be induced to differentiate into the neural cell type of choice using the appropriate media described in this invention. The drug to be tested can be added prior to differentiation to test for developmental inhibition, or added post-differentiation to monitor neural cell-type specific reactions.

The cells of this invention may be transplanted “naked” into patients according to conventional techniques, into the CNS, as described for example, in U.S. Pat. Nos. 5,082,670 and 5,618,531, each incorporated herein by reference, or into any other suitable site in the body.

In one embodiment, the human stem cells are transplanted directly into the CNS. Parenchymal and intrathecal sites are contemplated. It will be appreciated that the exact location in the CNS will vary according to the disease state.

Implanted cells may be labeled with bromodeoxyuridine (BrdU) prior to transplantation. As observed in various experiments, cells double stained for a neural cell marker and BrdU in the various grafts indicate differentiation of BrdU stained stem cells into the appropriate differentiated neural cell type (see Example 9). Transplantation of human forebrain derived neural stem cells to the hippocampus produced neurons that were predominantly NeuN staining but GABA negative. The NeuN antibody is known to stain neurons of the hippocampus. GABAergic neurons were formed when these same cell lines were transplanted into the striatum. Thus, transplanted cells respond to environmental clues in both the adult and the neonatal brain.

According to one aspect of this invention, provided herein is methodology for improving the viability of transplanted human neural stem cells. In particular, graft viability improves if the transplanted neural stem cells are allowed to aggregate, or to form neurospheres prior to implantation, as compared to transplantation of dissociated single cell suspensions. Preferably, small sized neurospheres are transplanted, approximately 10-500 μm in diameter, preferably 40-50 μm in diameter. Alternatively, spheres containing about 5-100, preferably 5-20 cells per sphere are preferred. A density of about 10,000-1,000,000 cells per μl, preferably 25,000-500,000 cells per μl, is preferred for transplantion.

The cells may also be encapsulated and used to deliver biologically active molecules, according to known encapsulation technologies, including microencapsulation (see, e.g., U.S. Pat. Nos. 4,352,883; 4,353,888; and 5,084,350, incorporated herein by reference),

(b) macroencapsulation (see, e.g., U.S. Pat. Nos. 5,284,761, 5,158,881, 4,976,859 and 4,968,733 and published PCT patent applications WO 92/19195, WO 95/05452, each incorporated herein by reference).

If the human neural stem cells are encapsulated, macroencapsulation as described in U.S. Pat. Nos. 5,284,761; 5,158,881; 4,976,859; 4,968,733; 5,800,828 and published PCT patent application WO 95/05452, each incorporated herein by reference is preferred. Cell number in the devices can be varied; preferably each device contains between 10³-10⁹ cells, most preferably 10⁵-10⁷ cells. A large number of macroencapsulation devices may be implanted in the patient; preferably between one to 10 devices.

In addition, “naked” transplantation of human stem cells in combination with a capsular device is also contemplated, wherein the capsular device secretes a biologically active molecule that is therapeutically effective in the patient or that produces a biologically active molecule that has a growth or trophic effect on the transplanted neural stem cells, or that induces differentiation of the neural stem cells towards a particular phenotypic lineage.

The invention further provides methods of inducing the in vivo migration and proliferation of progenitor cells transplanted to the brain. In one embodiment, in vivo migration of progenitor cells transplanted to a first locus of the brain of a subject is induced by infusing EGF at a second locus of the brain. In some preferred embodiments, the first locus is in the striatum of the brain, and the second locus at which EGF is infused is the lateral ventricle of the brain. In other preferred embodiments, EGF infusion induces migration towards the second locus (e.g., locus of infusion) but does not induce differentiation of the progenitor cells.

In another embodiment, in vivo proliferation of progenitor cells transplanted to a locus of the brain of a subject is induced by infusing EGF at or near the locus of transplantation. In some preferred embodiments, the locus of transplantation is in the striatum of the brain, and EGF is infused in the lateral ventricle of the brain. In other embodiments of the methods of the invention, the progenitor cells are mammalian embryonic progenitor cells, and the progenitor cells are cultured in media containing EGF prior to transplantation.

Any EGF-responsive neural stem cell suitable for treatment of a given neural disease state may be utilized. For example, EGF-responsive stem cells may be dissected from the striatal anlage, e.g., of transgenic embryonic mammals, such as mice. Progenitor cells may be cultured and propagated as described above. The cells may be cultured in growth medium containing EGF, and are prepared for transplantation by collecting small “spheres” of cells, typically of about 15-30 cells, as described above, by centrifugation and resuspending to a desired final concentration, typically 250,000 cells/μL. Progenitor cells may also be encapsulated for transplant, as described above.

Transplantation of cells to the brain of a subject is performed by stereotaxic surgery under anesthesia. Multiple deposits of cell sphere suspension may be made, for example 500,000 cells per deposit, in the striatum of the brain. After transplantation, an infusion cannulae is placed in the ventricle, e.g., lateral ventricle, for EGF infusion, and may be secured using dental cement. A minipump may be used to infuse EGF (e.g., dissolved in serum/gentamycin/saline solution) over a period of days. The total dose of EGF required to induce migration and proliferation of transplanted cells will vary somewhat from subject to subject, but may be, for example, around about 400 ng/day of EGF infused. Diving cells may be labeled for study by BrdU, for example by intraperitoneal injection of BrdU subsequent to cell transplantation. Alternatively, encapsulated EGF-producing cells may be implanted in the ventricle adjacent to the progenitor cell transplant.

EGF-responsive neural progenitor cells are able to respond to EGF after transplantation in vivo. Cells transplanted to the adult rat striatum are able to proliferate and migrate toward the source of intraventricular EGF and this response is maintained over the multiple days of EGF infusion. Some of these newly generated cells subsequently differentiate into glia, expressing the astrocytic marker GFAP. Newly generated BrdU-positive cells within the sub-ventricular zone (SVZ) may be found at a maximal distance of 1 mm rostral to the infusion cannulae, and not further away in the rostral migratory stream on route to the olfactory bulb. In addition, some cells remain at the site of proliferation, forming small nodules of SVZ which protrude into the lateral ventricle.

Transplanted progenitor cells show an active response to EGF in vivo, with proliferation and directed migration of cells away from the graft core toward the EGF source. EGF protein is able to penetrate and diffuse through the striatal parenchyma in order to exert an effect on the transplanted cells, which retain their responsiveness to EGF after transplantation in vivo. The present invention, therefore, provides for the intraventricular delivery of neural growth factors, e.g., EGF, as a promising system by which to manipulate cells after transplantation. The infusion of EGF in vivo provides a means to manipulate progenitor cells after transplantation, at least in the short term, to direct the cells towards specific differentiation, or directed migration, or to increase their survival. This technique will play an important role in overcoming problems associated with the limited migration and differentiation of transplanted cells, and therefore could increase the ability of transplanted neurons to reinnervate host tissue in neural transplantation paradigms.

No morphological differences are observed between grafted cells exposed to EGF in vivo and those that receive only vehicle infusions. Extensive glial differentiation is seen in all transplants, as evidenced by M2-positive profiles, whereas no neuronal differentiation is observed using either of the early neuronal markers Hu and β-III-tubulin. Therefore, it is likely that EGF exerts its effect on different types of cells within the mixed population found in these progenitor cell cultures, both on progenitors themselves and on more differentiated glial precursors.

The cells and methods of this invention may be useful in the treatment of various neurodegenerative diseases and other disorders. It is contemplated that the cells will replace diseased, damaged or lost tissue in the host. Alternatively, the transplanted tissue may augment the function of the endogenous affected host tissue. The transplanted neural stem cells may also be genetically modified to provide a therapeutically effective biologically active molecule.

Excitotoxicity has been implicated in a variety of pathological conditions including epilepsy, stroke, ischemia, and neurodegenerative diseases such as Huntington's disease, Parkinson's disease and Alzheimer's disease. Accordingly, neural stem cells may provide one means of preventing or replacing the cell loss and associated behavioral abnormalities of these disorders. Neural stem cells may replace cerebellar neurons lost in cerebellar ataxia, with clinical outcomes readily measurable by methods known in the medical arts.

Huntington's disease (HD) is an autosomal dominant neurodegenerative disease characterized by a relentlessly progressive movement disorder with devastating psychiatric and cognitive deterioration. HD is associated with a consistent and severe atrophy of the neostriatum, which is related to a marked loss of the GABAergic medium-sized spiny projection neurons, the major output neurons of the striatum. Intrastriatal injections of excitotoxins such as quinolinic acid (QA) mimic the pattern of selective neuronal vulnerability seen in HD. QA lesions result in motor and cognitive deficits, which are among the major symptoms seen in HD. Thus, intrastriatal injections of QA have become a useful model of HD and can serve to evaluate novel therapeutic strategies aimed at preventing, attenuating, or reversing neuroanatomical and behavioral changes associated with HD. Because GABAergic neurons are characteristically lost in Huntington's disease, treatment of Huntington's patients can be achieved by transplantation of cell cultures enriched in GABAergic neurons derived according to the methods of this invention.

Epilepsy is also associated with excitotoxicity. Accordingly, GABAergic neurons derived according to this invention are contemplated for transplantation into patients suffering from epilepsy.

The cells of the present invention can be used in the treatment of various demyelinating and dysmyelinating disorders, such as Pelizaeus-Merzbacher disease, multiple sclerosis, various leukodystrophies, post-traumatic demyelination, and cerebrovascular (CVS) accidents, as well as various neuritis and neuropathies, particularly of the eye. The present invention contemplates the use of cell cultures enriched in oligodendrocytes or oligodendrocyte precursor or progenitors, such cultures prepared and transplanted according to this invention to promote remyelination of demyelinated areas in the host.

The cells of the present invention can also be used in the treatment of various acute and chronic pains, as well as for certain nerve regeneration applications (such as spinal cord injury). The present invention also contemplates the use of human stem cells for use in sparing or sprouting of photoreceptors in the eye.

In another aspect of the present invention, the local delivery of a neurotrophic factor, such as EFG, to newly transplanted cells, in accordance with the invention, to provide a means of regulation in vivo, to guide undifferentiated progenitor cells to divide, migrate or differentiate into specific phenotypes, and may provide a controlled means to increase graft survival, reinnervation of host tissue and associated behavioral recovery, to enhance the effectiveness of transplantation as a potential restorative therapy for neurodegenerative diseases.

The cells and methods of this invention are intended for use in a mammalian host, recipient, patient, subject or individual, preferably a primate, most preferably a human.

All references cited herein are hereby incorporated by reference herein. The following examples are provided for illustrative purposes only, and are not intended to be limiting.

### EXAMPLES

### Example 1

### Media for Proliferating Neural Stem Cells

Proliferation medium was prepared with the following components in the indicated concentrations:

| COMPONENT | FINAL CONCENTRATION |
|---|---|
| 50/50 mix of DMEM/F-12 | 1X |
| glucose | 0.6% w/v |
| glutamine | 2 mM |
| NaHCO₃ | 3 mM |
| HEPES | 5 mM |
| apo-human transferrin (Sigma T-2252) | 100 μg/ml |
| human insulin (Sigma I-2767) | 25 μg/ml |
| putrescine (Sigma P-7505) | 60 μM |
| selenium (Sigma S-9133) | 30 nM |
| progesterone (Sigma P-6149) | 20 nM |
| human EGF (Gibco 13247-010) | 20 ng/ml |
| human bFGF (Gibco 13256-029) | 20 ng/ml |
| human LIF (R&D Systems 250-L) | 10 ng/ml |
| heparin (Sigma H-3149) | 2 μg/ml |

### Example 2

### Isolation of Human CNS Neural Stem Cells

Sample tissue from human embryonic forebrain was collected and dissected in Sweden and kindly provided by Huddinje Sjukhus. Blood samples from the donors were sent for viral testing. Dissections were performed in saline and the selected tissue was placed directly into proliferation medium (as described in Example 1). Tissue was stored at 4° C. until dissociated. The tissue was dissociated using a standard glass homogenizer, without the presence of any digesting enzymes. The dissociated cells were counted and seeded into flasks containing proliferation medium. After 5-7 days, the contents of the flasks are centrifuged at 1000 rpm for 2 min. The supernatant was aspirated and the pellet resuspended in 200 μl of proliferation medium. The cell clusters were triturated using a P200 pipetman about 100 times to break up the clusters. Cells were reseeded at 75,000-100,000 cells/ml into proliferation medium. Cells were passaged every 6-21 days depending upon the mitogens used and the seeding density. Typically these cells incorporate BrdU, indicative of cell proliferation. For T75 flask cultures (initial volume 20 ml), cells are “fed” 3 times weekly by addition of 5 ml of proliferation medium. In a preferred embodiment, Nunc flasks are used for culturing.

Nestin Staining for Proliferating Neurospheres

Cells were stained for nestin (a measure of proliferating neurospheres) as follows. Cells were fixed for 20 min at room temperature with 4% paraformaldehyde. Cells were washed twice for 5 min with 0.1 M PBS, pH 7.4. Cells were permeabilized for 2 min with 100% EtOH. The cells were then washed twice for 5 min with 0.1 M PBS. Cell preparations were blocked for 1 hr at room temperature in 5% normal goat serum (“NGS”) diluted in 0.1 M PBS, pH 7.4 and 1% Triton X-100 (Sigma X-100) for 1 hr at room temperature with gentle shaking. Cells were incubated with primary antibodies to human nestin (from Dr. Lars Wahlberg, Karolinska, Sweden, rabbit polyclonal used at 1:500) diluted in 1% NGS and 1% Triton X-100 for 2 hr at room temperature. Preparations were then washed twice for 5 min with 0.1 M PBS. Cells were incubated with secondary antibodies (pool of GAM/FITC used at 1:128, Sigma F-0257; GAR/TRITC used at 1:80, Sigma T-5268) diluted in 1% NGS and 1% Triton X-100 for 30 min at room temperature in the dark. Preparations are washed twice for 5 min with 0.1 M PBS in the dark. Preparations are mounted onto slides face down with mounting medium (Vectashield Mounting Medium, Vector Labs., H-1000) and stored at 4° C.

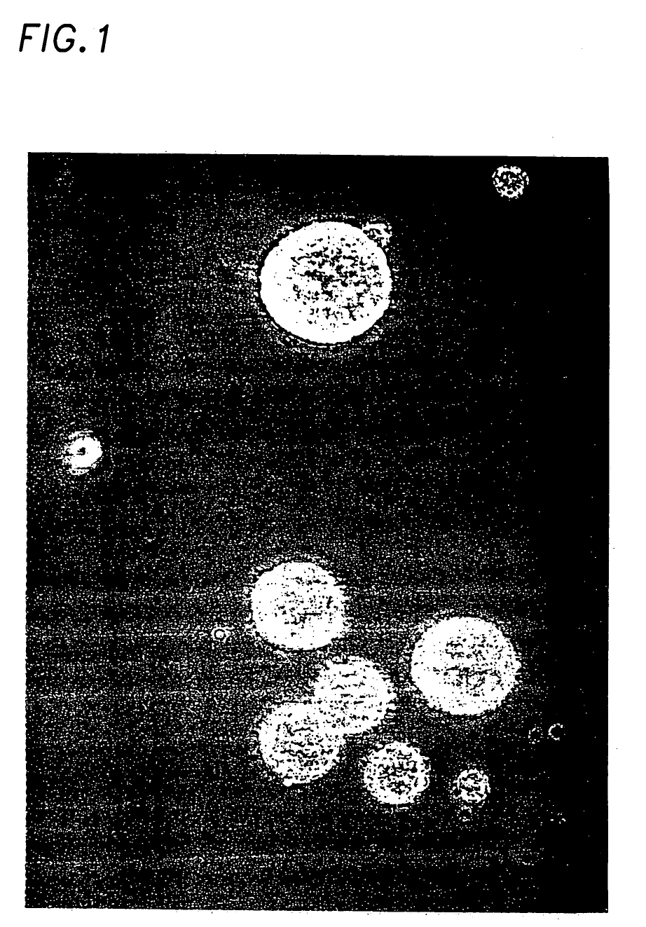

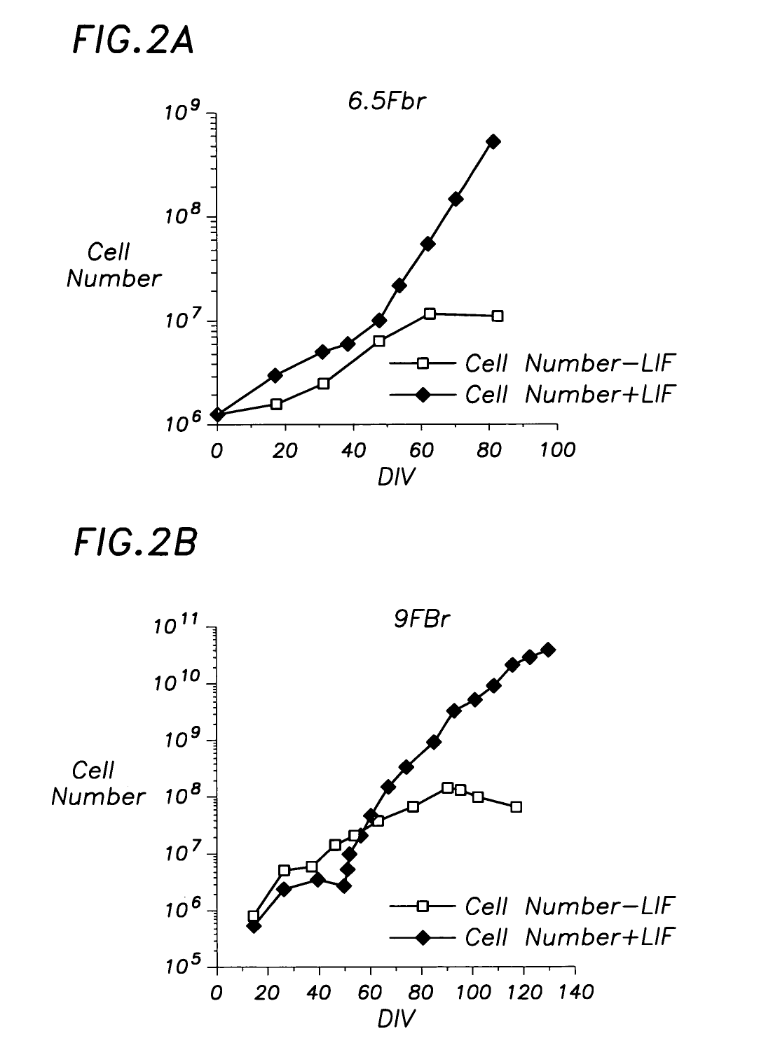

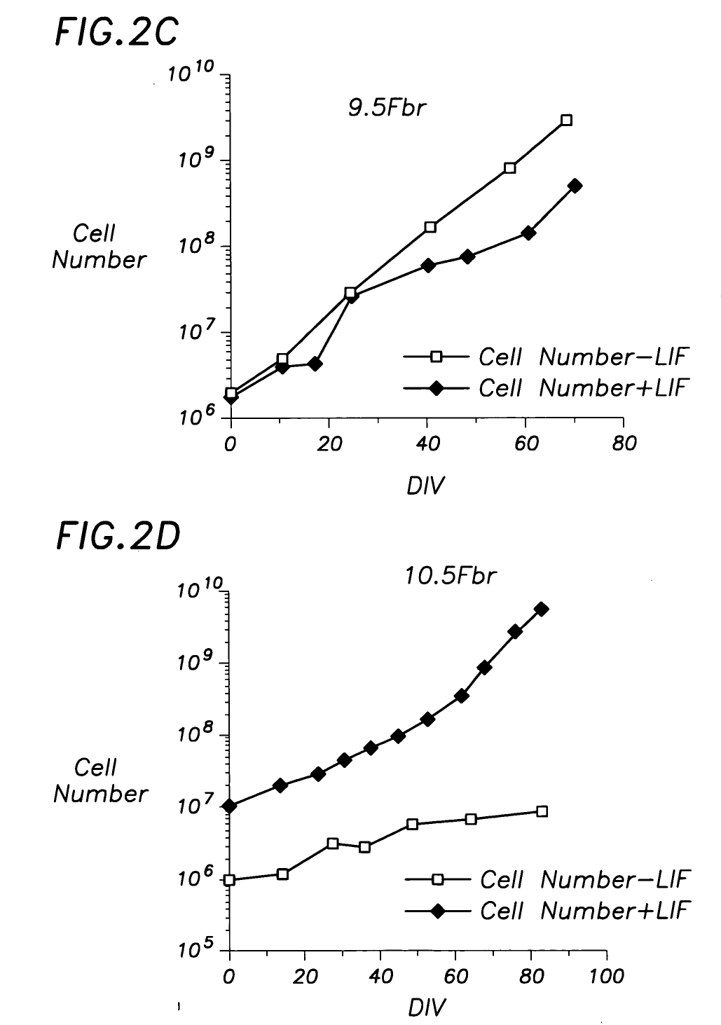

FIG. 1 shows a picture of proliferating spheres (here called “neurospheres”) of human forebrain derived neural stem cells. The proliferation of four lines of human forebrain derived neural stem cells were evaluated in proliferation medium as described above with LIF present of absent. As illustrated in FIG. 2, LIF significantly increased the rate of cell proliferation in three of the four lines (6.5Fbr, 9Fbr, and 10.5FBr). The effect of LIF was most pronounced after about 60 days in vitro.

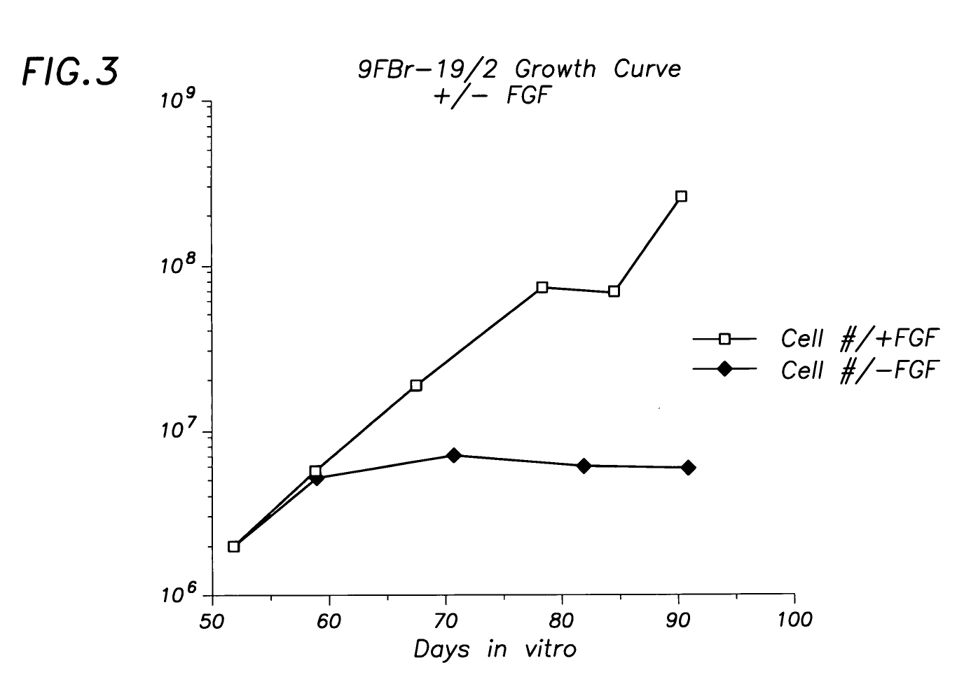

The effect of bFGF on the rate of proliferation of human forebrain-derived neural stem cells were also evaluated. As FIG. 3 illustrates, the stem cells proliferation was significantly enhanced in the presence of bFGF.

### Example 3

### Differentiation of Human Neural Stem Cells

In a first differentiation protocol, the proliferating neurospheres were induced to differentiate by removal of the growth factor mitogens and LIF, and provision of 1% serum, a substrate and a source of ionic charges (e.g., glass cover slip covered with poly-omithine).

The staining protocol for neurons, astrocytes and oligodendrocytes was as follows:

β-tubulin Staining for Neurons

Cells were fixed for 20 min at room temperature with 4% paraformaldehyde. Cells were washed twice for 5 min with 0.1 M PBS, pH 7.4. Cells were permeabilized for 2 min with 100% EtOH. The cells were then washed twice for 5 min with 0.1 M PBS. Cell preparations were blocked for 1 hr at room temperature in 5% normal goat serum (“NGS”) diluted in 0.1 M PBS, pH 7.4. Cells were incubated with primary antibodies to β-tubulin (Sigma T-8660, mouse monoclonal; used at 1:1,000) diluted in 1% NGS for 2 hr at room temperature. Preparations were then washed twice for 5 min with 0.1 M PBS. Cells were incubated with secondary antibodies (pool of GAM/FITC used at 1:128, Sigma F-0257; GAR/TRITC used at 1:80, Sigma T-5268) diluted in 1% NGS for 30 min at room temperature in the dark. Preparations are washed twice for 5 min with 0.1 M PBS in the dark. Preparations are mounted onto slides face down with mounting medium (Vectashield Mounting Medium, Vector Labs., H-1000) and stored at 4° C.

In some instances, cells were also stained with DAPI (a nuclear stain) as follows. Coverslips prepared as above are washed with DAPI solution (diluted 1:1000 in 100% MeOH, Boehringer Mannheim, #236276). Coverslips are incubated in DAPI solution for 15 min at 37° C.

O4 Staining for Oligodendrocytes

Cells were fixed for 10 min at room temperature with 4% paraformaldehyde. Cells were washed three times for 5 min with 0.1 M PBS, pH 7.4. Cell preparations were blocked for 1 hr at room temperature in 5% normal goat serum (“NGS”) diluted in 0.1 M PBS, pH 7.4. Cells were incubated with primary antibodies to O4 (Boehringer Mannheim # 1518 925, mouse monoclonal; used at 1:25) diluted in 1% NGS for 2 hr at room temperature. Preparations were then washed twice for 5 min with 0.1 M PBS. Cells were incubated with secondary antibodies, and further processed as described above for β-tubulin.

GFAP Staining for Astrocytes

Cells were fixed for 20 min at room temperature with 4% paraformaldehyde. Cells were washed twice for 5 min with 0.1 M PBS, pH 7.4. Cells were permeabilized for 2 min with 100% EtOH. The cells were then washed twice for 5 min with 0.1 M PBS. Cell preparations were blocked for 1 hr at room temperature in 5% normal goat serum (“NGS”) diluted in 0.1 M PBS, pH 7.4. Cells were incubated with primary antibodies to GFAP (DAKO Z 334, rabbit polyclonal; used at 1:500) diluted in 1% NGS for 2 hr at room temperature. Preparations were then washed twice for 5 min with 0.1 M PBS. Cells were incubated with secondary antibodies, and further processed as described above for β-tubulin.

This differentiation protocol produced cell cultures enriched in neurons as follows:

| | | | | % of neurons |
|---|---|---|---|---|
| | | % GFAP | % β-tubulin | that are |
| Cell Line | Passage | Positive | positive | GABA positive |
| 6.5FBr | 5 | 15 | 37 | 20 |
| 9FBr | 7 | 52 | 20 | 35 |
| 10.5FBr | 5 | 50 | 28 | 50 |

The ability of a single cell line to differentiate consistently as the culture aged (i.e., at different passages) was also evaluated using the above differentiation protocol. The data are as follows:

| | | % GFAP | % β-tubulin | % of neurons that are |
|---|---|---|---|---|
| Cell Line | Passage | Positive | positive | GABA positive |
| 9FBr | 5 | 53 | 20.4 | ND |
| 9FBr | 9 | ND | 20.3 | 34.5 |
| 9FBr | 15 | 62 | 17.9 | 37.9 |

These data suggests that cells will follow reproducible differentiation patterns irrespective of passage number or culture age.

### Example 4

### Differentiation of Human Neural Stem Cells

In a second differentiation protocol, the proliferating neurospheres were induced to differentiate by removal of the growth factor mitogens and LIF, and provision of 1% serum, a substrate (e.g., glass cover slip or extracellular matrix components), a source of ionic charges (e.g., poly-ornithine) as well as a mixture of growth factors including 10 ng/ml PDGF A/B, 10 ng/ml CNTF, 10 ng/ml IGF-1, 10 μM forskolin, 30 ng/ml T3, 10 ng/ml LIF and 1 ng/ml NT-3. This differentiation protocol produced cell cultures highly enriched in neurons (i.e., greater than 35% of the differentiated cell culture) and enriched in oligodendrocytes.

### Example 5

### Differentiation of Human Neural Stem Cells

In a third differentiation protocol, cell suspensions were initially cultured in a cocktail of hbFGF, EGF, and LIF, were then placed into altered growth media containing 20 ng/mL hEGF (GIBCO) and 10 ng/mL human leukemia inhibitory factor (hLIF) (R&D Systems), but without hbFGF. The cells initially grew significantly more slowly than the cultures that also contained hbFGF (see FIG. 3). Nonetheless, the cells continued to grow and were passaged as many as 22 times. Stem cells were removed from growth medium and induced to differentiate by plating on poly-omithine coated glass coverslips in differentiation medium supplemented with a growth factor cocktail (hPDGF A/B, hCNTF, hGF-1, forskolin, T3 and hNT-3). Surprisingly, GalC immunoreactivity was seen in these differentiated cultures at levels that far exceeded the number of O4 positive cells seen in the growth factor induction protocol described in Example 4.

Hence, this protocol produced differentiated cell cultures enrichment in oligodendrocytes. Neurons were only occasionally seen, had small processes, and appeared quite immature.

### Example 6

### Genetic Modification

A glioblast cell line derived from the human neural stem cells described herein was conditionally immortalized using the Mx-1 system described in WO 96/02646. In the Mx-1 system, the Mx-1 promoter drives expression of the SV40 large T antigen. The Mx-1 promoter is induced by interferon. When induced, large T is expressed, and quiescent cells proliferate.

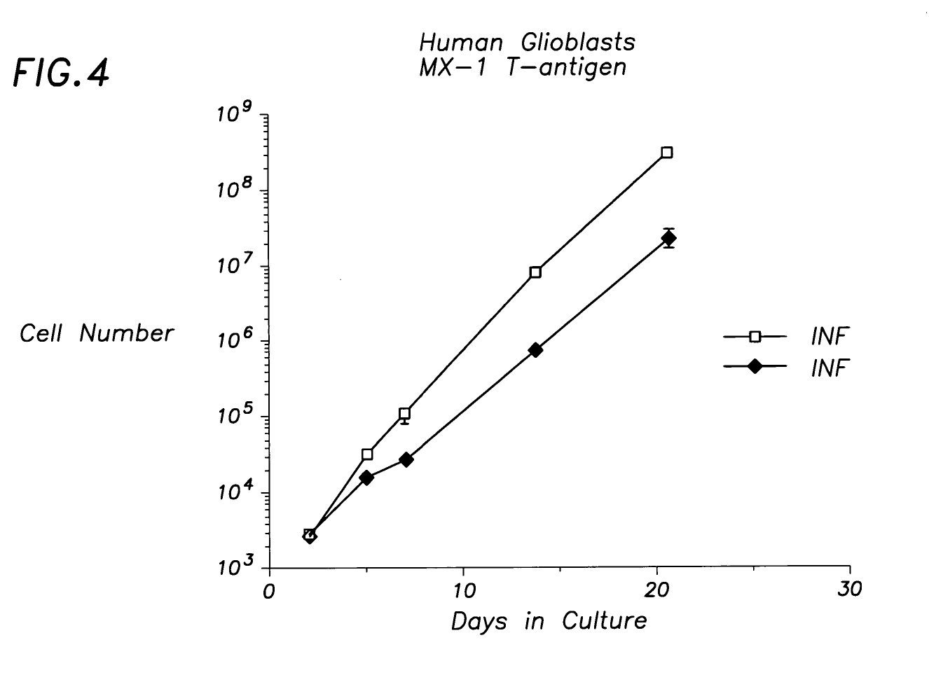

Human glioblasts were derived from human forebrain neural stem cells as follows. Proliferating human neurospheres were removed from proliferation medium and plated onto poly-ornithine plastic (24 well plate) in a mixture of N2 with the mitogens EGF, bFGF and LIF, as well as 0.5% FBS. 0.5 ml of N2 medium and 1% FBS was added. The cells were incubated overnight. The cells were then transfected with p318 (a plasmid containing the Mx-1 promoter operably linked to the SV 40 large T antigen) using Invitrogen lipid kit (lipids 4 and 6). The transfection solution contained 6 μl/ml of lipid and 4 μl/ml DNA in optiMEM medium. The cells were incubated in transfection solution for 5 hours. The transfection solution was removed and cells placed into N2 and 1% FBS and 500 U/ml A/D interferon. The cells were fed twice a week. After ten weeks cells were assayed for large T antigen expression. The cells showed robust T antigen staining at this time. As FIG. 4 shows, cell number was higher in the presence of interferon than in the absence of interferon.

Large T expression was monitored using immunocytochemistry as follows. Cells were fixed for 20 min at room temperature with 4% paraformaldehyde. Cells were washed twice for 5 min with 0.1 M PBS, pH 7.4. Cells were permeabilized for 2 min with 100% EtOH. The cells were then washed twice for 5 min with 0.1 M PBS. Cell preparations were blocked for 1 hr at room temperature in 5% normal goat serum (“NGS”) diluted in 0.1 M PBS, pH 7.4. Cells were incubated with primary antibodies to large T antigen (used at 1:10) diluted in 1% NGS for 2 hr at room temperature. An antibody to large T antigen was prepared by culturing PAB 149 cells and obtaining the conditioned medium. Preparations were then washed twice for 5 min with 0.1M PBS. Cells were incubated with secondary antibodies (goat-anti-mouse biotinylated at 1:500 from Vector Laboratories, Vectastain Elite ABC mouse IgG kit, PK-6102) diluted in 1% NGS for 30 min at room temperature. Preparations are washed twice for 5 min with 0.1 M PBS. Preparations are incubated in ABC reagent diluted 1:500 in 0.1 M PBS, pH 7.4 for 30 min at room temperature. Cells are washed twice for 5 min in 0.1 M PBS, pH 7.4, then washed twice for 5 min in 0.1 M Tris, pH 7.6. Cells are incubated in DAB (nickel intensification) for 5 min at room temperature. The DAB solution is removed, and cells are washed three to five times with dH2O. Cells are stored in 50% glycerol/50% 0.1 M PBS, pH 7.4.

### Example 7

### Encapsulation

If the human neural stem cells are encapsulated, then the following procedure may be used:

The hollow fibers are fabricated from a polyether sulfone (PES) with an outside diameter of 720 μm and a wall thickness of a 100 μm (AKZO-Nobel Wüppertal, Germany). These fibers are described in U.S. Pat. Nos. 4,976,859 and 4,968,733, herein incorporated by reference. The fiber may be chosen for its molecular weight cutoff. In a preferred embodiment, a PES#5 membrane with a MWCO of about 280 kd is used. In another preferred embodiment, a PES#8 membrane with a MWCO of about 90 kd is used.

The devices typically comprise:

1) a semipermeable poly (ether sulfone) hollow fiber membrane fabricated by AKZO Nobel Faser AG;

2) a hub membrane segment;

3) a light cured methacrylate (LCM) resin leading end; and

4) a silicone tether.

The semipermeable membrane used typically has the following characteristics:

| | |
|---|---|
| Internal Diameter | 500 ± 30 μm |
| Wall Thickness | 100 ± 15 μm |
| Force at Break | 100 ± 15 cN |
| Elongation at Break | 44 ± 10% |
| Hydraulic Permeability | 63 ± 8 (ml/min m² mm Hg) |
| nMWCO (dextrans) | 280 ± 20 kd |

The components of the device are commercially available. The LCM glue is available from Ablestik Laboratories (Newark, Del.); Luxtrak Adhesives LCM23 and LCM24). The tether material is available from Specialty Silicone Fabricators (Robles, Calif.). The tether dimensions are 0.79 mm OD×0.43 mm ID×length 202 mm. The morphology of the device is as follows: The inner surface has a permselective skin. The wall has an open cell foam structure. The outer surface has an open structure, with pores up to 1.5 μm occupying 30±5% of the outer surface.

Fiber material is first cut into 5 cm long segments and the distal extremity of each segment sealed with a photopolymerized acrylic glue (LCM-25, ICI). Following sterilization with ethylene oxide and outgassing, the fiber segments are loaded with a suspension of between 10⁴⁻10⁷ cells, either in a liquid medium, or a hydrogel matrix (e.g., a collagen solution (Zyderm®), alginate, agarose or chitosan) via a Hamilton syringe and a 25 gauge needle through an attached injection port. The proximal end of the capsule is sealed with the same acrylic glue. The volume of the device contemplated in the human studies is approximately 15-18 μl.

A silicone tether (Specialty Silicone Fabrication, Taunton, Mass.) (ID: 690 μm; OD: 1.25 mm) is placed over the proximal end of the fiber allowing easy manipulation and retrieval of the device.

### Example 8

### Transplantation of Neural Stem Cells

Human neural stem cells were transplanted into rat brain and assessed graft viability, integration, phenotypic fate of the grafted cells, as well as behavioral changes associated with the grafted cells in lesioned animals.

Transplantation was performed according to standard techniques. Adult rats were anesthetized with sodium pentobarbitol (45 mg/kg, i.p.) And positioned in a Kopf stereotaxic instrument. A midline incision was made in the scalp and a hole drilled for the injection of cells. Rats received implants of unmodified, undifferentiated human neural stem cells into the left striatum using a glass capillary attached to a 10 μl Hamilton syringe. Each animal received a total of about 250,000-500,000 cells in a total volume of 2 μl. Cells were transplanted 1-2 days after passaging and the cell suspension was made up of undifferentiated stem cell clusters of 5-20 cells. Following implantation, the skin was sutured closed.

Animals were behaviorally tested and then sacrificed for histological analysis.

### Example 9

### Intraventricular EGF Delivery with Transplantation of Neural Stem Cells

Approximately 300,000 neural stem cells were transplanted as small neurospheres into the adult rat striatum close to the lateral ventricle using standard techniques. During the same surgery session, osmotic minipumps releasing either EGF (400 ng/day) or vehicle were also implanted in the striatum. The rats received EGF over a period of 7 days at a flow rate of 0.5 μL/hr, resulting in the delivery of 2.8 μg EGF in total into the lateral ventricle of each animal. Subsets of implanted rats were additionally immunosuppressed by i.p. cyclosporin injections (10 mg/kg/day). During the last 16 hours of pump infusion, the animals received injections of BrdU every three hours (120 mg/kg).

One week after transplantation, the animals were perfused with 4% para-formaldehyde and serial sections cut on a freezing microtome at 30 μm thickness. Brain sections were stained for astrocytes, oligodendrocytes, neuron, and undifferentiated progenitor cell markers. Minimal migration was demonstrated in adult CNS in the absence of EGF. Excellent survival of the 7 day old grafts was seen in rats receiving EGF as demonstrated by M2 immunoreactivity, and grafts in EGF-treated animals were more extensive than in animals treated with vehicle alone. Furthermore, proliferation of host cells was observed upon EGF treatment. Animals receiving BrdU injections before sacrifice demonstrated an increased number of dividing cells in the treated ventricle, but not the adjoining ventricles.

### Example 10

### Treatment of Syringomyelia

Primary fetal transplants have been used to obliterate the syrinx formed around spinal cord injuries in patients. The neural stem cells described in this invention are suitable for replacement, because only a structural function would be required by the cells. Neural stem cells are implanted in the spinal cord of injured patients to prevent syrinx formation. Outcomes are measured preferably by MRI imaging. Clinical trial protocols have been written and could easily be modified to include the described neural stem cells.

### Example 11

### Treatment of Neurodegenerative Disease Using Progent of Human Neural Stem Cells Prolifereated in Vitro

Cells are obtained from ventral mesencephalic tissue from a human fetus aged 8 weeks following routine suction abortion, which is collected into a sterile collection apparatus. A 2×4×1 mm piece of tissue is dissected and dissociated as in Example 2. Neural stem cells are then proliferated. Neural stem cell progeny are used for neurotransplantation into a blood-group matched host with a neurodegenerative disease. Surgery is performed using a BRW computed tomographic (CT) stereotaxic guide. The patient is given local anesthesia suppiemencea with intravenously administered midazolam. The patient undergoes CT scanning to establish the coordinates of the region to receive the transplant. The injection cannula consists of a 17-gauge stainless steel outer cannula with a 19-gauge inner stylet. This is inserted into the brain to the correct coordinates, then removed and replaced with a 19-gauge infusion cannula that has been preloaded with 30 μl of tissue suspension. The cells are slowly infused at a rate of 3 μl/min as the cannula is withdrawn. Multiple stereotactic needle passes are made throughout the area of interest, approximately 4 mm apart. The patient is examined by CT scan postoperatively for hemorrhage or edema. Neurological evaluations are performed at various post-operative intervals, as well as PET scans to determine metabolic activity of the implanted cells.

### Example 12

### Genetic Modification of Neural Stem Cell Progeny Using Calcium Phosphate Transfection

Neural stem cell progeny are propagated as described in Example 2. The cells are then transfected using a calcium phosphate transfection technique. For standard calcium phosphate transfection, the cells are mechanically dissociated into a single cell suspension and plated on tissue culture-treated dishes at 50% confluence (50,000-75,000 cells/cm²) and allowed to attach overnight.

The modified calcium phosphate transfection procedure is performed as follows: DNA (15-25 μg) in sterile TE buffer (10 mM Tris, 0.25 mM EDTA, pH 7.5) diluted to 440 μl with TE, and 60 μl of 2M CaCl₂ (pH to 5.8 with 1 M HEPES buffer) is added to the DNA/TE buffer. A total of 500 μl of 2×HeBS (HEPES-Buffered saline; 275 mM NaCl, 10 mM KCl, 1.4 mM Na₂HPO₄, 12 mM dextrose, 40 mM HEPES buffer powder, pH 6.92) is added dropwise to this mix. The mixture is allowed to stand at room temperature for 20 minutes. The cells are washed briefly with 1× HeBS and 1 ml of the calcium phosphate precipitated DNA solution is added to each plate, and the cells are incubated at 37° C. for 20 minutes. Following this incubation, 10 ml of complete medium is added to the cells, and the plates are placed in an incubator (37° C., 9.5% CO₂) for an additional 3-6 hours. The DNA and the medium are removed by aspiration at the end of the incubation period, and the cells are washed 3 times with complete growth medium and then returned to the incubator.

### Example 13

### Genetic Modification of Neural Stem Cell Progeny

Cells proliferated as in Examples 2 are transfected with expression vectors containing the genes for the FGF-2 receptor or the NGF receptor. Vector DNA containing the genes are diluted in 0.1 X TE (1 mM Tris pH 8.0, 0.1 mM EDTA) to a concentration of 40 μg/ml. 22 μl of the DNA is added to 250 μl of 2×HBS (280 mM NaCl, 10 mM KCl, 1.5 mM Na₂HPO₄2H₂O, 12 mM dextrose, 50 mM HEPES) in a disposable, sterile 5 ml plastic tube. 31 μl of 2M CaCl₂ is added slowly and the mixture is incubated for 30 minutes at room temperature. During this 30 minute incubation, the cells are centrifuged at 800 g for 5 minutes at 4° C. The cells are resuspended in 20 volumes of ice-cold PBS and divided into aliquots of 1×107 cells, which are again centrifuged. Each aliquot of cells is resuspended in 1 ml of the DNA-CaCl₂ suspension, and incubated for 20 minutes at room temperature. The cells are then diluted in growth medium and incubated for 6-24 hours at 37° C. in 5%-7% CO₂. The cells are again centrifuged, washed in PBS and returned to 10 ml of growth medium for 48 hours.

The transfected neural stem cell progeny are transplanted into a human patient using the procedure described in Example 8 or Example 11, or are used for drug screening procedures as described in the example below.

### Example 14

### Screening of Drugs or Other Biological Agents for Effects on Multipotent Neural Stem Cells and Neural Stem Cell Progeny

A. Effects of BDNF on Neuronal and Glial Cell Differentiation and Survival

Precursor cells were propagated as described in Example 2 and differentiated as described in Example 4. At the time of plating the cells, BDNF was added at a concentration of 10 ng/ml. At 3, 7, 14, and 21 days in vitro (DIV), cells were processed for indirect immunocytochemistry. BrdU labeling was used to monitor proliferation of the neural stem cells. The effects of BDNF on neurons, oligodendrocytes and astrocytes were assayed by probing the cultures with antibodies that recognize antigens found on neurons (MAP-2, NSE, NF), oligodendrocytes (O4, GalC, MBP) or astrocytes (GFAP). Cell survival was determined by counting the number of immunoreactive cells at each time point and morphological observations were made. BDNF significantly increased the differentiation and survival of neurons over the number observed under control conditions. Astrocyte and oligodendrocyte numbers were not significantly altered from control values.

B. Effects of BDNF on the Differentiation of Neural Phenotypes

Cells treated with BDNF according to the methods described in Part A were probed with antibodies that recognize neural transmitters or enzymes involved in the synthesis of neural transmitters. These included TH, ChAT, substance P, GABA, somatostatin, and glutamate. In both control and BDNF-treated culture conditions, neurons tested positive for the presence of substance P and GABA. As well as an increase in numbers, neurons grown in BDNF showed a dramatic increase in neurite extension and branching when compared with control examples.

C. Identification of Growth-Factor Responsive Cells

Cells were differentiated as described in Example 4, and at 1 DIV approximately 100 ng/ml of BDNF was added. At 1, 3, 6, 12 and 24 hours after the addition of BDNF the cells were fixed and processed for dual label immunocytochemistry. Antibodies that recognize neurons (MAP-2, NSE, NF), oligodendrocytes (O4, GalC, MBP) or astrocytes (GFAP) were used in combination with an antibody that recognizes c-fos and/or other immediate early genes. Exposure to BDNF resulted in a selective increase in the expression of c-fos in neuronal cells.

D. Effects of BDNF on the Expression of Markers and Regulatory Factors During Proliferation and Differentiation

Cells treated with BDNF according to the methods described in Part A are processed for analysis of the expression of regulatory factors, FGF-R1 or other markers.

E. Effects of Chlorpromazine on the Proliferation, Differentiation, and Survival of Growth Factor Generated Stem Cell Progeny

Chlorpromazine, a drug widely used in the treatment of psychiatric illness, is used in concentrations ranging from 10 ng/ml to 1000 ng/ml in place of BDNF in Examples 14A to 14D above. The effects of the drug at various concentrations on stem cell proliferation and on stem cell progeny differentiation and survival is monitored. Alterations in gene expression and electrophysiological properties of differentiated neurons are determined.

### Example 15

### Induction of In Vivo Proliferation and Migration of Transplanted Progenitor Cells in the Brain

In order to investigate whether EGF-responsive murine progenitor cells would remain responsive to intraventricularly administered EGF after their transplantation in vivo, embryonic cells generated from transgenic mice carrying the beta-galactosidase enzyme (lacZ) gene under the control of the promoter for myelin basic protein (MBP), and grown in medium containing EGF, were transplanted in the medial striatum of the adult rat. EGF was administered over seven days after transplantation to assess its affects on the proliferation migration and differentiation of the transplanted cells.

Cell Source

EGF-responsive stem cells were generated from transgenic mice containing the insertion of the β-galactosidase enzyme under the control of the MPB promoter (MPB-lacZ). The striatal anlage was dissected from el4.5-e 15.5 mouse embryos as described previously. See Reynolds et al., Journal ofNeuroscience 12, pp. 4565-4574 (1992). The pieces of tissue were broken up into a single cell suspension by mechanical trituration using a flame-polished pasteur pipette, and the cells resuspended growth medium: N2, a defined DMEM:F12-based GIBCO medium containing 0.6% glucose, 25 μg/ml insulin, 100 μg/ml transferrin, 20 nM progesterone, 60 pM putrescine, 30 nM selenium chloride, 2 nM glutamine, 3 mM sodium bicarbonate, 5 mM HEPES and 20 ng/ml human recombinant epidermal growth factor (EGF, R & D Systems). The cells grew as free-floating clusters or “spheres”, and were passaged by trituration to a single cell suspension every seven days.

Preparation of Cells for Transplantation

After 5 weeks in culture the cells were pr4ared for transplantation. ³H-Thymidine, 2.5 μCi/ml, was added to the cultures on days 1 and 3 after the final passage. On day 5 after passage the small spheres of typically 15-30 cells were collected by centrifugation and resuspended to a final concentration of 250,000 cells/μl. Viability was checked using trypan blue exclusion and revealed approximately 90% viable cells within the spheres.

Surgery

Adult female Sprague-Dawley rats weighing approximately 220 g were used in this study. The animals were maintained in a temperature and humidity controlled environment with a 12-hour light/dark cycle and ad libitum food and water throughout the experiment. Three experimental groups were included in the study: EGF-infusion with immunosuppression (n=8), EGF-infusion without immunosuppression (n=6) and vehicle infusion with immunosuppression (n=6). Animals receiving immunosuppression obtained daily intraperitoneal injections of cyclosporin, 10 mg/kg, beginning on the day of transplantation.

Stereotaxic surgery was performed under deep equithesin anesthesia (3 ml/kg body weight, i.p.). Each rat received six deposits of 0.3 μl sphere suspension, equivalent to approximately 500,000 cells, at the following coordinates: AP=+0.4, L=−2.0, V=−4.5, −4.0, −3.5; AP=+0.0, L=−2.0, V=−4.5, −4.0, −3.5.

Immediately after transplantation, a steel infusion cannula was placed in position in the ventricle (coordinates: AP=+0.2, L=−1.2, V=−3.5) and secured using dental cement. The extracranial end of the cannula was attached to a minipump device (Alzet, 1007D, infusion rate 0.5:1/hour), placed dorsally under the skin of the neck. Infusion was over 7 days with either 400 ng/day EGF dissolved in a solution of 0.1% rat serum and 0.01% gentamycine in 0.9% saline, or control vehicle without EGF. This gave a total delivery of 3.2 μg EGF during the study.

BrdU Labeling of Dividing Cells in Situ

Seven days after transplantation each rat received repeated intraperitoneal injections of 120 mg/kg BrdU in sterile saline every three hours, beginning 16 hours prior to perfusion.

Histology

One hour after the final injection, the rats were terminally anaesthetized with an overdose of chloral hydrate, and transcardially perfused with 0.1M phosphate buffered saline (PBS) followed by 250 ml 4% paraformaldehyde in PBS, over 5 minutes. The brains were removed and immersed in 4% paraformaldehyde overnight before being rinsed and transferred to a 25% sucrose solution in PBS.

The brains were cut on a freezing microtome at a thickness of 3 μm. Fluorescence immunohistochemistry was performed on series of sections, for different combinations of markers. Free floating sections were preincubated in blocking solution of potassium phosphate buffered saline (KPBS) containing 5% normal donkey serum (NDS) and 5% normal rabbit serum (NRS) for one hour. This solution was then replaced with the primary antibodies, made up in blocking solution, for 36 hours at 4° C. For M2, no triton was included in the procedure. antibodies used in this study were: M2 (a mouse-specific glial marker a gift from Dr. Carl Lagenhauer) 1:50; BrdU (Calbiochem) 1:100, or (Beckton Dickinson) 1:25; glial fibrillary acidic protein (GFAP, Dakopatts) 1:500; VIM (Dakopatts) 1:25; nestin (a gift from Dr. Ron McKay) 1:50; β-tubulin-III (Sigma, St Louis, Mo.) 1:400; Hu (a gift from Dr. S. Goldman) 1:2000. After three rinses in KPBS, the sections were incubated with the appropriate secondary antibodies (donkey anti-mouse secondary conjugated to FITC, 1:200 (Jackson); donkey anti-mouse secondary conjugated to Cy3, 1:200 (Jackson); donkey anti-rat secondary conjugated to FIT C, 1:200 (Jackson); donkey anti-mouse secondary conjugated to Cy5, 1:200 (Jackson); donkey anti-rabbit secondary conjugated to FITC, 1:200 (Jackson); biotinylated rabbit anti-rat 1:200 (DAKO); in KPBS with 2% of the appropriate normal sera, for 2 hours at room temperature, in the dark. After three further rinses, and where a biotinylated secondary antibody was used, the final incubation was in streptavidin conjugated to Cy3, in -KPBS, for 2 hours at room temperature in the dark. Sections were mounted on chromalum coated glass slides, dried for 5 minutes in air and coverslipped using PVA/DABCO mountant.

A further series of sections were stained for BrdU, but with diamino-benzidine (DAB) as the chromogen. These were mounted, delipidized and dipped in Kodak50 emulsion for 6 weeks to assess thymidine labeling. The sections were then counterstained with cresyl violet before dehydrating and coverslipping with DPX. Using a similar immunohistochemistry protocol, expression of the lacZ transgene was investigated, using an antibody to β-galactosidase (βgal, 1:500, 5′3′Inc.).

Analysis

Fluorescent sections were viewed in a Bio-Rad MRC1024UV confocal scanning microscope to enable exact definition of each of the antibodies. Double-labeled cells were verified by collecting serial sections of 1-2 μm throughout the specimen.

Volumes of the graft cores were measured using M2 positivity. A full series of 1:8 sections was taken through each graft, and the area of the densely stained graft core was outlined in each section, and the area calculated using an image analysis system. The areas were then converted to volumes for comparison, using a standard ANOVA test (Statview software).

Results

All animals had good surviving transplants as observed with M2 immunoreactivity and ³H-thymidine labeling. There was a clear effect of EGF infusion on the transplanted cells, both in their increased migration toward the source of EGF and in their proliferation. Therefore transplanted murine progenitors were able to respond to EGF in vivo, in the same manner that they respond under culture conditions.

1. Host Response to EGF Infusion

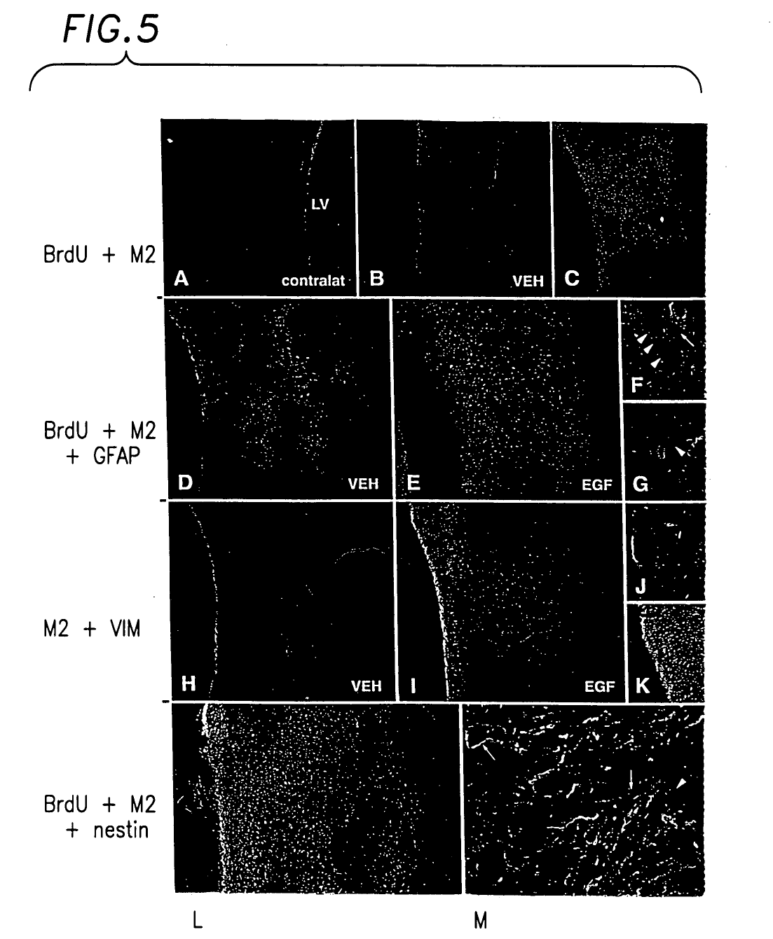

Continued injections of BrdU to the host animals for 16 hours prior to perfusion revealed good labeling of the endogenous population of cells situated in the SVZ adjacent to the lateral ventricle. On the side contralateral to the cannula placement a few BrdU-positive nuclei were observed scattered in a single layer adjacent to the ventricular wall (FIG. 5A). These were seen in both vehicle and EGF-treated animals. In animals which received pump infusions of vehicle alone there was a slight increase in the number of BrdU-positive nuclei in the ipsilateral subventricular region of the lateral wall overlying, the striatum. The cells were observed throughout 4-5 layers, with a few scattered nuclei up to 1 mm lateral to the ventricular wall (FIGS. 5B,D). However in the animals which received infusions of EGF a significant number of BrdU-positive nuclei were observed in 12-15 cell layers adjacent to the ventricle, with many more positive cells scattered throughout the striatum lateral to the infusion site (FIGS. 1 C,E,L). The host response to EGF infusion with increased numbers of BrdU-positive cells was confined to the lateral ventricular wall and was observed in the SVZ for up to 1 mm rostral and caudal to the cannula placement. In addition, nodules of SVZ had appeared, jutting in to the ventricular space. These were filled with BrdU-positive nuclei (e.g., see FIG. 5E top).

The astrocyte marker, 3FAP was used to identify the host glial reaction to the EGF infusion. In vehicle-infused animals that also received a transplant, GFAP reactive astrocytes were observed in the periphery of the transplant core, intermingled with M2-positive profiles (FIG. 5D). In BGF-infused animals, the GFAP-positivity was more extensive and individual cells were observed scattered in the region between the transplant and the lateral ventricle as well as surrounding and within the transplant core itself (FIG. 5E). High power microscopy revealed that a number of the GFAP-positive cells were also labeled with BrdU, both within the graft core (FIG. 5F) and in the area of striatum between the transplant and lateral ventricle (FIG. 5G), indicating that these cells had divided in the last 16 hours prior to perfusion.

Vimentin (VIM)-positivity was used to delineate both the immature cells of the SVZ and reactive immature astrocytes present in the host striatum. In vehicle-infused animals VIM staining of immature cells was restricted to the SVZ and scattered immature astrocytes surrounding the graft core (FIG. 5H). In EGF-infused animals the VIM-positive SVZ appeared thickened (FIG. 5I), indicative of an increase in cell number, with extension of radial-like VIM-positive processes emanating from the SVI into the adjacent striatum (FIG. 5K). Slightly further away from the SVZ (200-400 μm), individual immature VIM-positive glia were observed (FIG. 5J).

The antibody nestin was used as a marker of immature progenitor cells. See Lendahl et al., Cell 60, pp. 585-595 (1990). Nestin immunoreactivity showed a similar distribution to vimentin. In vehicle-infused animals nestin-positive cells were restricted to the SVZ, while in EGF-infused this region was thickened indicative of cell division (FIG. 5L). In addition, in animals receiving EGF, numerous nestin-positive profiles were observed in the region between the lateral ventricle and the transplants. High power microscopy revealed double-labeled. BrdU/nestin-positive cells within this area (FIG. 5M).

2. Graft Response to EGF Infusion

All animals had nice surviving grafts revealed using the mouse-specific astrocyte marker M2 (FIG. 5). The majority of grafts were placed in the striatum in close proximity to the lateral ventricle. However, in two animals some of the graft tissue had been misplaced in the ventricle itself and was seen attached to the ventricle wall. In all EGF-infused animals the dense M2-positive core of the grafts appeared to be within a similar range in volume. Graft volume did not differ between the EGF-infused animals, which received cyclosporin, and those, which did not, indicating that neither did the non-immunosuppressed animals show any form of graft rejection during the survival period, nor did administration of cyclosporin alter the effectiveness of EGF on the transplanted cells. Therefore the non-immunosuppressed animals have been included in the EGF-infused group for all analysis of the results.

(a) Migration of Cells Towards the Lateral Ventricle

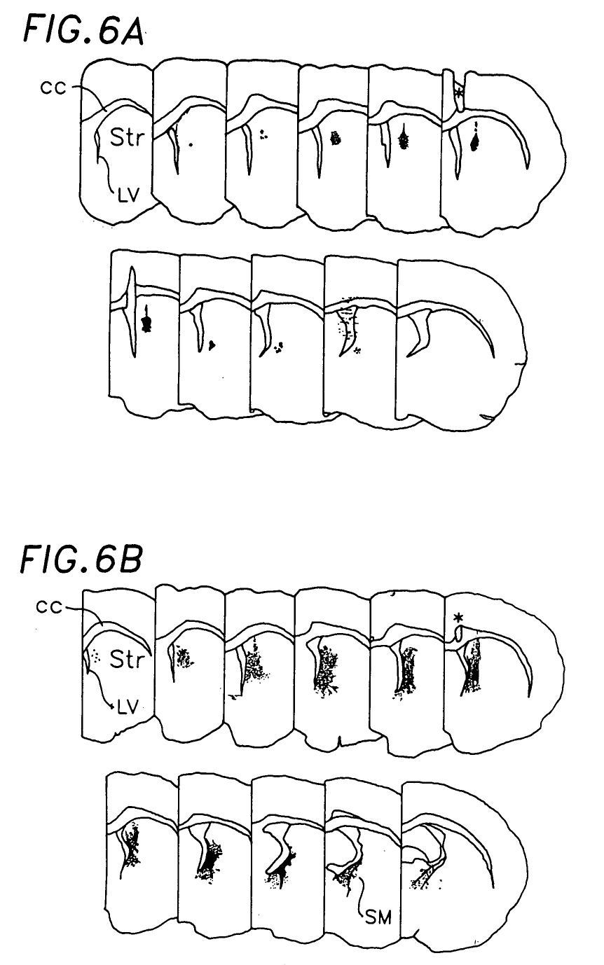

In the vehicle-infused animals (FIGS. 5B,D,H, and FIG. 6A), the grafts were characteristically dense with very little M2-posltive staining outside the graft core. M2-positive profiles were observed emanating from the graft in all directions to a limited extent into the surrounding parenchyma (FIGS. 5 B,D,H).

In the EGF-infused animals there was a striking pattern of M2-positive staining outside the graft core only on the side toward the lateral ventricle (FIGS. 5 C,E,I,L and FIG. 6B). There was a significant increase in the number of profiles stained with M2, and these were found throughout the parenchyma as far as the ventricular wall itself. In some animals there was an increase in M2 positivity in the SVZ, with many M2-positive profiles densely packed within this area. In addition, many M2-positive profiles within the region between the graft and SVZ were seen to be oriented towards the lateral ventricle (FIGS. 5 I,L). On the side distal to the ventricle very little M2-positive staining was observed outside the graft core.

Expression of the M2 marker was observed for up to 1 mm rostral and caudal to the graft (FIG. 6). In more caudal sections from EGF-infused animals, the profiles were observed in the white matter tracts of the stria medullaris (SM in FIG. 6B), running parallel to the axonal profiles.

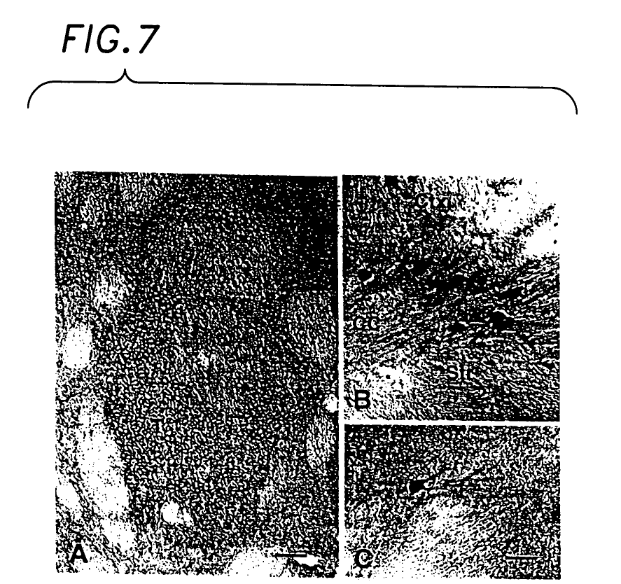

A series of sections which was first stained for BrdU was dipped in emulsion for six weeks to look for ³H-Thymidine expression of the grafted cells. In all animals, autoradiographic grains were observed throughout the graft core (FIG. 7). Immediately surrounding the graft, scattered cells could be identified containing numerous silver grains over the nucleus. In vehicle-infused animals these were only observed in the area immediately surrounding the graft and not in the zone between the grafted cells and the lateral ventricle (FIG. 7B). However, in the EGF-infused animals scattered ³H-Thymidine positive cells were seen throughout the parenchyma on the side of the graft adjacent to the ventricle, as far as the SVZ (FIG. 7A, arrows).

(b) Proliferation of Grafted Cells

To assess whether the graft population was dividing in response to EGF, the BrdU/³H-Thymidine double-labeled sections were assessed for colocalization of these two markers. The majority of BrdU-labeled cells in the zone between the graft deposit and lateral ventricle did not contain a significant number of silver grains, above background levels. However, scattered BrdU/³H-Thymidine double-labeled cells were occasionally observed (arrowhead in FIG. 7A). In addition, fluorescence immunohistochemistry showed there was an increase in the number of BrdU-positive cells found within the M2-positive area in the EGF-infused animals. BrdU/M2 double-labeled cells could be found in the graft core (FIG. 5F), and in the region between the transplant and lateral ventricle (FIGS. 5G,M). Not all BrdU-labeled cells were double-labeled with M2, however a proportion of these were positive for GFAP as described above, and the remainder did not label with either M2 or GFAP. In the vehicle-infused animals BrdU-positive cells were often found interspersed with GFAP or M2-positive profiles, with only a few occasional cells double-labeled for either marker.

Further evidence for proliferation of the transplanted cells within the EGF-infused group was obtained from the analysis of graft volumes. Each graft volume was calculated by measuring the dense M2-positive graft core through one series of sections, excluding the regions of scattered M2-positive profiles in the EGF-infused groups. This analysis showed the volume of the graft core was similar in animals which had received EGF or vehicle infusions compared to controls, (vehicle-infused=0.81±0.2; EGF-infused=1.1 5±0.57; p>0.05), indicating that the increase in dispersed M2-positive profiles outside the core i.e., in the region adjacent to the lateral ventricle in the EGF-infused group was not due solely to the migration of cells away from the graft core, but also in part due to proliferation of the grafted cells.

(c) Graft Morphology

There were no obvious differences in the morphology of the transplanted cells when comparing vehicle or EGF-infused animals. Immunohistochemistry in all animals revealed many M2-positive profiles indicating a large number cells with glial morphology (FIG. 5). There was an overlap of GFAP and M2-positive staining, with profiles intermingled in these areas (FIGS. 5D-G). Although overlapping GFAP and M2 profiles were observed, closer analysis of Z series through the tissue sections did not reveal co-localized expression of these two markers. This was also the case with M2 and VIM. Although M2-positive profiles were often observed intermingled with VIM-positive glia, no double-labeled cells were observed. Therefore, those populations of M2-positive but GFAP or VIM-negative cells are assumed to be immature or non-reactive glia that do not express GFAP or VIM. The transplants were also stained with the mouse-specific marker, M6 that stains both neurons and a subset of astrocytes. See e.g., Campbell et al., Neuron 15, pp. 1259-1273 (1995). There was completely overlapping expression of M6 with areas of M2 positivity. No M6-positive profiles with neuronal morphology were observed.

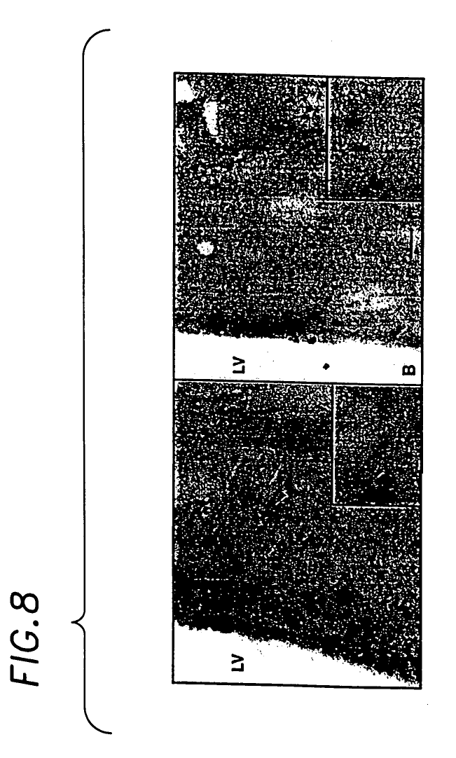

The cells used in this study were derived from transgenic mice carrying the β-galactosidase enzyme (lacZ) under the myelin basic protein (MBP) promoter. Sections were stained for β-galactosidase (βgal) to look for expression of the gene. In all animals, regardless of infusion media, there was very low βgal expression within the graft core. Where positive staining was seen at this site, the expression was punctuate, giving the cells a spherical immature appearance (FIG. 8K). In cases where positive βgal staining was observed away from the graft core, good expression was seen throughout the cells and primary processes. Cells found in the grey matter had a relatively immature morphology, with either uni- or bipolar extensions (FIG. 8C). In one case where cells were found in the needle tract at the level of the corpus callosum, these cells had more extensive processes elongating in the same orientation as the axonal profiles of the host (FIG. 8B), and had the morphology of immature oligodendrocytes.

Transplants were also analyzed for expression of the early neuronal markers Hu (4) and β-III-tubulin, in combination with the M2 antibody in each case. No cells positive for either of these antibodies were found either within the transplant region itself or in the region between the transplant core and the lateral ventricle (data not shown).

The above-described results suggest that EGF-responsive murine neural progenitor cells are able to respond to EGF after transplantation in vivo. Cells transplanted to the adult rat striatum are able to proliferate and migrate toward the source of intraventricular EGF and this response is maintained over the 7 days of EGF infusion.

As previously observed (Craig et al., supra.; Kuhn et al., supra.), infusion of EGF to the lateral ventricle stimulates division of SVZ progenitor cells and their migration into the surrounding striatum. The current study shows at 7 days from the start of EGF infusion, that some of these newly generated cells differentiated into glia, expressing the astrocytic marker GFAP Newly generated BrdU-positive cells within the SVZ were found at a maximal distance of 1 mm rostral to the infusion cannulae and not further away in the rostral migratory stream on route to the olfactory bulb. In addition, some cells remained at the site of proliferation, forming small nodules of SVZ that protruded into the lateral ventricle. This correlates with previous reports that EGF infusion prevents the active migration of SVZ progenitor cells in their normal route toward the olfactory bulb (Kuhn et al., supra; Threadgill, et al., Science 269, pp. 230-234 (1995)), and promotes their differentiation into a glial rather than neuronal phenotype.

Transplanted murine progenitor cells showed an active response to EGF in vivo, with proliferation and directed migration of cells away from the graft core toward the EGF source. Two conclusions that can be drawn from these results are that the EGF protein is able to penetrate and diffuse through the striatal parenchyma in order to exert an effect on the transplanted cells, and that the murine cells retained their responsiveness to EGF even after transplantation in vivo. It is possible that addition of neurotrophic factors (see e.g., Ahmed et al., supra; Kirschenbaum et al., Cerebral Cortex 6, pp. 576-589 (1994)) in vivo may provide a means to manipulate progenitor cells after transplantation, at least in the short term, to direct the cells towards specific differentiation, or directed migration, or to increase their survival. This technique could play an important role in overcoming problems associated with the limited migration and differentiation of transplanted cells, and therefore could increase the ability of transplanted neurons to reinnervate host tissue in neural transplantation paradigms. It appears that there is a threshold level of EGF required to affect the migration and differentiation of transphiMed progenitor cells. Studies combining encapsulated EGF-secreting cells placed in the ventricle adjacent to EGF-responsive progenitor cell transplants had no effect on the migrational capacity of these cells (unpublished observations). The amount of EGF secreted by the cell lines was 100 times less that the cannula infusion and had no effect on the endogenous progenitor cells, suggesting that this level is insufficient to elicit a response.

No morphological differences were observed between the grafted cells that were exposed to EGF in vivo and those that received vehicle infusions. Extensive glial differentiation was seen in all transplants as evidenced by M2-positive profiles, whereas no neuronal differentiation was observed using either of the early neuronal markers Hu and β-III-S tubulin. Therefore, it is likely that EGF exerts its effect on different types of cells within the mixed population found in these progenitor cell cultures, both on progenitors themselves and on more differentiated glial precursors.

Evidence for EGF acting on more mature glial-restricted progenitors comes from previous studies where EGF has been shown to support glial-restricted but not neuron-18 restricted progenitors both in vitro and in vivo (Kilpatrick et al., J. Neuroscience 15, pp. 3653-3661 (1995); Kuhn et al., supra). Indeed, both of the antibodies used to identify glia in this study, GFAP and M2, are known to label more mature glial cells, with spheres of progenitor cells being negative for both markers (See, e.g., Winkler et al., Neuroscience 11, pp. 99-116 (1998)). Once these progenitors are induced to differentiate in vitro, cells that adopt a glial phenotype express GFAP and/or M2. In culture, the expression of M2 co-localizes with GFAP, however, not all M2-positive cells are also GFAP-positive. This population of M2-positive/GFAP-negative cells could account for the grafted cells, which are double-labeled with BrdU and M2, but do not express GFAP. Indeed, our previous studies indicate that the expression of GFAP and M2 is not co-localized in murine progenitor cells after transplantation in vivo (Winkler et al., supra.).

Previous studies have shown that although murine EGF-responsive progenitor cells are multipotent in vitro; they differentiate preferentially into a glial phenotype after transplantation in either the developing or adult rat brain. See e.g., Hammang et al., Experimental Neurology 147, pp. 84-95 (1997); Winkler et al., Molecular and Cellular Neuroscience 11, pp. 99-116 (1998). In this study, double-labeling with BrdU and M2 revealed newly generated murine glial cells in the animals which received EGF infusions when compared to the vehicle-infused group, suggesting that EGF stimulated the division of those transplanted progenitors which were committed to a glial phenotype. It is likely, therefore, that the EGF infusion stimulated cell division and migration, but not differentiation of the grafted cells, in vivo in a similar manner to its actions in vitro.

A number of BrdU-positive cells within the graft area did not express either M2 or GFAP. These cells may belong to one of two populations, either host progenitor cells, or transplanted progenitors, both of which have a more undifferentiated, immature phenotype. It is possible that EGF may also play a role in maintaining the transplanted donor cells in progenitor-like state, similar to its role in culture. See, e.g., Reynolds et al., Developmental Biology 175, pp. 1-13 (1996).

A third population of cells found within the graft and region adjacent to the lateral ventricle could be double-labeled with BrdU and GFAP, but not with M2. It is likely that this population represents newly divided glial cells which originate from the host SVZ, as we have previously observed that all GFAP-positive murine progenitors simultaneously express M2 after their differentiation in vitro. See e.g., Winkler et al. (1998), supra. Further evidence for this may come from BrdU/nestin double-labeled cells found within the region between the transplant and the lateral ventricle. These cells may have been derived from either the murine or host progenitor cells, which have divided in response to the EGF infusion.

The cells used in this transplantation study were obtained from transgenic mice, therefore carried the transgene lacZ under the control of the MBP promoter. Expression of β-galactosidase, as a sign of oligodendrocyte formation in vivo, revealed a small number of transplanted cells with an immature oligodendrocyte morphology, mainly within the white matter tracts, e.g., the corpus callosum. The small number of lacZ-positive cells found within the transplants, suggests that the majority of the cells had differentiated into astrocytes rather than oligodendrocytes as has been seen previously. See e.g., Winkler et al. (1998), supra.; Winkler et al., Society for Neuroscience Abstracts (1995).

However, the presence of lacZ-positive cells within the rat brain indicates that the transgene can still be expressed under appropriate conditions after xenotransplantation, and supports the efficacy of using these cells as a tool to enable the introduction of relevant genes to the brain. It remains to be seen whether more differentiated oligodendrocytes are observed after longer survival times.

These results indicate that neural growth factor infusion can stimulate murine progenitor cells in vivo, after transplantation to the adult rat brain. This technique of local delivery of a neurotrophic factor to newly transplanted cells, provides a novel means of regulation in vivo, to guide undifferentiated progenitor cells to proliferate, migrate or differentiate into specific phenotypes, and further provides a controlled means to increase graft survival, reinnervation of host tissue and associated behavioral recovery, to enhance the effectiveness of transplantation as a potential restorative therapy for neurodegenerative diseases.

### EQUIVALENTS

From the foregoing detailed description of the specific embodiments of the present invention, it should be readily apparent that unique methods for inducing in vivo proliferation and migration of transplanted progenitor cells in the brain have been described herein. Although particular embodiments have been disclosed herein in detail, this has been done by way of example for purposes of illustration only, and is not intended to be limiting with respect to the scope of the appended claims and equivalents thereto which follow. In particular, it is contemplated by the inventors that various substitutions, alterations, and modifications may be made to the invention without departing from the spirit and scope of the invention as defined by the claims. For instance, the choice of the particular mitogenic growth factor is believed to be a matter of routine for a person of ordinary skill in the art with knowledge of the embodiments described herein.

We claim:

1. A method for screening drugs or biological agents which affect proliferation, differentiation or survival of human neural stem cells, comprising:
(a) transplanting said human neural stem cells to a first locus of the CNS of a non-human mammal selected from the group consisting of rats and mice;
(b) contacting said non-human mammal with at least one drug or biological agent, and
(c) determining if said at least one drug or biological agent has an effect on proliferation, differentiation or survival of said human neural stem cells.

2. The method of claim 1 wherein step (c) comprises determining the effects of said biological agent on differentiation of said human neural stem cells.

3. The method of claim 1 further comprising the step of inducing differentiation of said human neural stem cells prior to performing step (b).

4. The method of claim 1 wherein the effect of the at least one drug or biological agent on proliferation of the human neural stem cells determined by observing changes in size or number of the neurospheres.

5. A non-human animal useful for screening drugs or biological agents which affect proliferation, differentiation or survival of human neural stem cells, wherein the non-human animal is selected from the group consisting of rats and mice and wherein the non-human animal has human neural stem cells integrated into its CNS.

6. The non-human animal of claim 5, wherein the human neural stem cells are transplanted to a first locus of the CNS of the non-human mammal, wherein the transplanted neural stem cells migrate in vivo after implantation from the first locus to other anatomic sites for integration within the CNS of the non-human mammal following infusion of a mitogenic growth factor that does not induce differentiation of the human neural stem cells at a second locus of the CNS, and wherein the implanted neural stem cells integrate into the parenchymal tissues at a local anatomic site in the non-human mammal.

7. A method for screening drugs or biological agents which affect proliferation, differentiation or survival of human neural stem cells, comprising:
(a) contacting the non-human mammal of claim 5 with at least one drug or biological agent, and
(b) determining if said at least one drug or biological agent has an effect on proliferation, differentiation or survival of said human neural stem cells.
